SEQUENCE LISTING (35 sequences)

SEQ ID NO: 1 - DNA, 2926 bp, Homo sapiens
  source 1..2926: mol_type = genomic DNA
agaagaaaacagttccacgttgcttgaaattgaaaatcaagataaaaatgttcacaattaagctccttctttttattgttcctctagttatttcctccagaattgatcaagacaattcatcatttgattctctatctccagagccaaaatcaagatttgctatgttagacgatgtaaaaattttagccaatggcctccttcagttgggacatggtcttaaagactttgtccataagacgaagggccaaattaatgacatatttcaaaaactcaacatatttgatcagtctttttatgatctatcgctgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgaaactccagaacacccagaagtaacttcacttaaaacttttgtagaaaaacaagataatagcatcaaagaccttctccagaccgtggaagaccaatataaacaattaaaccaacagcatagtcaaataaaagaaatagaaaatcagctcagaaggactagtattcaagaacccacagaaatttctctatcttccaagccaagagcaccaagaactactccctttcttcagttgaatgaaataagaaatgtaaaacatgatggcattcctgctgaatgtaccaccatttataacagaggtgaacatacaagtggcatgtatgccatcagacccagcaactctcaagtttttcatgtctactgtgatgttatatcaggtagtccatggacattaattcaacatcgaatagatggatcacaaaacttcaatgaaacgtgggagaactacaaatatggttttgggaggcttgatggagaattttggttgggcctagagaagatatactccatagtgaagcaatctaattatgttttacgaattgagttggaagactggaaagacaacaaacattatattgaatattctttttacttgggaaatcacgaaaccaactatacgctacatctagttgcgattactggcaatgtccccaatgcaatcccggaaaacaaagatttggtgttttctacttgggatcacaaagcaaaaggacacttcaactgtccagagggttattcaggaggctggtggtggcatgatgagtgtggagaaaacaacctaaatggtaaatataacaaaccaagagcaaaatctaagccagagaggagaagaggattatcttggaagtctcaaaatggaaggttatactctataaaatcaaccaaaatgttgatccatccaacagattcagaaagctttgaatgaactgaggcaaatttaaaaggcaataatttaaacattaacctcattccaagttaatgtggtctaataatctggtattaaatccttaagagaaagcttgagaaatagattttttttatcttaaagtcactgtctatttaagattaaacatacaatcacataaccttaaagaataccgtttacatttctcaatcaaaattcttataatactatttgttttaaattttgtgatgtgggaatcaattttagatggtcacaatctagattataatcaataggtgaacttattaaataacttttctaaataaaaaatttagagacttttattttaaaaggcatcatatgagctaatatcacaactttcccagtttaaaaaactagtactcttgttaaaactctaaacttgactaaatacagaggactggtaattgtacagttcttaaatgttgtagtattaatttcaaaactaaaaatcgtcagcacagagtatgtgtaaaaatctgtaatacaaatttttaaactgatgcttcattttgctacaaaataatttggagtaaatgtttgatatgatttatttatgaaacctaatgaagcagaattaaatactgtattaaaataagttcgctgtctttaaacaaatggagatgactactaagtcacattgactttaacatgaggtatcactataccttatttgttaaaatatatactgtatacattttatatattttaacacttaatactatgaaaacaaataattgtaaaggaatcttgtcagattacagtaagaatgaacatatttgtggcatcgagttaaagtttatatttcccctaaatatgctgtgattctaatacattcgtgtaggttttcaagtagaaataaacctcgtaacaagttactgaacgtttaaacagcctgacaagcatgtatatatgtttaaaattcaataaacaaagacccagtccctaaattatagaaatttaaattattcttgcatgtttatcgacatcacaacagatccctaaatccctaaatccctaaagattagatacaaattttttaccacagtatcacttgtcagaatttatttttaaatatgattttttaaaactgccagtaagaaattttaaattaaacccatttgttaaaggatatagtgcccaagttatatggtgacctacctttgtcaatacttagcattatgtatttcaaattatccaatatacatgtcatatatatttttatatgtcacatatataaaagatatgtatgatctatgtgaatcctaagtaaatattttgttccagaaaagtacaaaataataaaggtaaaaataatctataattttcaggaccacagactaagctgtcgaaattaacgctgatttttttagggccagaataccaaaatggctcctctcttcccccaaaattggacaatttcaaatgcaaaataattcattatttaatatatgagttgcttcctctatttggtttcc

SEQ ID NO: 2 - RNA, 21 nt, synthetic construct
  source 1..21: mol_type = other RNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
gctcaacatatttgatcagta

SEQ ID NO: 3 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide with phosphorothioate linkage
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  modified_base 20: mod_base = OTHER; note = 2'-fluoro modified nucleotide with phosphorothioate linkage
  modified_base 21: mod_base = OTHER; note = 2'-O-methyl modified nucleotide
tactgatcaaatatgttgagc

SEQ ID NO: 4 - RNA, 19 nt, synthetic construct
  source 1..19: mol_type = other RNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
ctacatataaactacaagt

SEQ ID NO: 5 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide
acttgtagtttatatgtag

SEQ ID NO: 6 - RNA, 21 nt, synthetic construct
  source 1..21: mol_type = other RNA
  modified_base 1: mod_base = OTHER; note = 2'-fluoro modified nucleotide
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
  modified_base 20: mod_base = OTHER; note = 2'-O-methyl modified nucleotide
  modified_base 21: mod_base = OTHER; note = 2'-fluoro modified nucleotide
tacatataaactacaagtcaa

SEQ ID NO: 7 - RNA, 23 nt, synthetic construct
  source 1..23: mol_type = other RNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  modified_base 20: mod_base = OTHER; note = 2'-fluoro modified nucleotide
  modified_base 21: mod_base = OTHER; note = 2'-O-methyl modified nucleotide with phosphorothioate linkage
  modified_base 22: mod_base = OTHER; note = 2'-fluoro modified nucleotide with phosphorothioate linkage
  modified_base 23: mod_base = OTHER; note = 2'-O-methyl modified nucleotide
ttgacttgtagtttatatgtagt

SEQ ID NO: 8 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-fluoro modified nucleotide
  modified_base 18: mod_base = OTHER; note = 2'-fluoro modified nucleotide
  modified_base 19: mod_base = OTHER; note = 2'-O-methyl modified nucleotide
  modified_base 20: mod_base = OTHER; note = Nucleotide with phosphorothioate linkage
  misc_feature 1..19: note = RNA
  misc_feature 20..21: note = DNA
ctacatataaactacaagttt

SEQ ID NO: 9 - DNA, 21 bp, synthetic construct
  source 1..21: mol_type = other DNA
  modified_base 1: mod_base = OTHER; note = 2'-fluoro modified nucleotide
  modified_base 2: mod_base = OTHER; note = 2'-fluoro modified nucleotide
  modified_base 3: mod_base = OTHER; note = 2'-fluoro modified nucleotide
  modified_base 4: mod_base = OTHER; note = 2'-fluoro modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-fluoro modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-fluoro modified nucleotide
  modified_base 20: mod_base = OTHER; note = Nucleotide with phosphorothioate linkage
  misc_feature 1..19: note = RNA
  misc_feature 20..21: note = DNA
acttgtagtttatatgtagtt

SEQ ID NO: 10 - RNA, 21 nt, synthetic construct
  source 1..21: mol_type = other RNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
gctcaacatatttgatcagta

SEQ ID NO: 11 - RNA, 19 nt, synthetic construct
  source 1..19: mol_type = other RNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
ctacatataaactacaagt

SEQ ID NO: 12 - DNA, 25 bp, synthetic construct
  source 1..25: mol_type = other DNA
agagcaccaagaactactccctttc

SEQ ID NO: 13 - DNA, 28 bp, synthetic construct
  source 1..28: mol_type = other DNA
catgatggcattcctgctgaatgtacca

SEQ ID NO: 14 - DNA, 24 bp, synthetic construct
  source 1..24: mol_type = other DNA
tgccacttgtatgttcacctctgt

SEQ ID NO: 15 - DNA, 25 bp, synthetic construct
  source 1..25: mol_type = other DNA
acgtgccacttatatgttcacctct

SEQ ID NO: 16 - DNA, 22 bp, synthetic construct
  source 1..22: mol_type = other DNA
agatgtaggccgggtgatcttt

SEQ ID NO: 17 - DNA, 31 bp, synthetic construct
  source 1..31: mol_type = other DNA
tgttccaaaaacagtggataattttgtggcc

SEQ ID NO: 18 - DNA, 24 bp, synthetic construct
  source 1..24: mol_type = other DNA
gtagccaaatcctttctctcctgt

SEQ ID NO: 19 - RNA, 21 nt, synthetic construct
  source 1..21: mol_type = other RNA
  modified_base 1: mod_base = OTHER; note = 2'-fluoro modified nucleotide
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
  modified_base 20: mod_base = OTHER; note = 2'-O-methyl modified nucleotide
  modified_base 21: mod_base = OTHER; note = 2'-fluoro modified nucleotide attached to N-[tris(N -acetylgalactosamine-alkyl)-amidodecanoyl)]-4-hydroxyproli nol (Hyp-(GalNAc-alkyl)3)
tacatataaactacaagtcaa

SEQ ID NO: 20 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-fluoro modified nucleotide
  modified_base 18: mod_base = OTHER; note = 2'-fluoro modified nucleotide
  modified_base 19: mod_base = OTHER; note = 2'-O-methyl modified nucleotide
  modified_base 20: mod_base = OTHER; note = Nucleotide with phosphorothioate linkage
  modified_base 21: mod_base = OTHER; note = Nucleotide attached to N-[tris(N-acetylgalactosamin e-alkyl)-amidodecanoyl)]-4-hydroxyprolinol (Hyp-(GalNAc-alkyl)3)
  misc_feature 1..19: note = RNA
  misc_feature 20..21: note = DNA
ctacatataaactacaagttt

SEQ ID NO: 21 - DNA, 39 bp, synthetic construct
  source 1..39: mol_type = other DNA
tttgaattcgccaccatgttcacaattaagctccttctt

SEQ ID NO: 22 - DNA, 35 bp, synthetic construct
  source 1..35: mol_type = other DNA
tacattgctagctcattcaaagctttctgaatctg

SEQ ID NO: 23 - DNA, 21 bp, synthetic construct
  source 1..21: mol_type = other DNA
tcatgcctctttgcaccattc

SEQ ID NO: 24 - DNA, 20 bp, synthetic construct
  source 1..20: mol_type = other DNA
aatggcctccttcagttggg

SEQ ID NO: 25 - DNA, 21 bp, synthetic construct
  source 1..21: mol_type = other DNA
gccaagagcaccaagaactac

SEQ ID NO: 26 - DNA, 21 bp, synthetic construct
  source 1..21: mol_type = other DNA
cacttcaactgtccagagggt

SEQ ID NO: 27 - DNA, 22 bp, synthetic construct
  source 1..22: mol_type = other DNA
tgtcttgatcaattctggagga

SEQ ID NO: 28 - DNA, 20 bp, synthetic construct
  source 1..20: mol_type = other DNA
attggtcttccacggtctgg

SEQ ID NO: 29 - DNA, 24 bp, synthetic construct
  source 1..24: mol_type = other DNA
tgtttgttgtctttccagtcttcc

SEQ ID NO: 30 - DNA, 19 bp, synthetic construct
  source 1..19: mol_type = other DNA
ccttgctccataccacccc

SEQ ID NO: 31 - DNA, 6623 bp, synthetic construct
  source 1..6623: mol_type = other DNA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatatcaagctttaatagtaatcaattacggggtcattagttcatagcccatatatggagttccgaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtggtttagtggatatccttaagggcccagccggcccgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgcgagaattcgccaccatgttcacaattaagctccttctttttattgttcctctagttatttcctccagaattgatcaagacaattcatcatttgattctctatctccagagccaaaatcaagatttgctatgttagacgatgtaaaaattttagccaatggcctccttcagttgggacatggtcttaaagactttgtccataagacgaagggccaaattaatgacatatttcaaaaactcaacatatttgatcagtctttttatgatctatcgctgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgaaactccagaacacccagaagtaacttcacttaaaacttttgtagaaaaacaagataatagcatcaaagaccttctccagaccgtggaagaccaatataaacaattaaaccaacagcatagtcaaataaaagaaatagaaaatcagctcagaaggactagtattcaagaacccacagaaatttctctatcttccaagccaagagcaccaagaactactccctttcttcagttgaatgaaataagaaatgtaaaacatgatggcattcctgctgaatgtaccaccatttataacagaggtgaacatacaagtggcatgtatgccatcagacccagcaactctcaagtttttcatgtctactgtgatgttatatcaggtagtccatggacattaattcaacatcgaatagatggatcacaaaacttcaatgaaacgtgggagaactacaaatatggttttgggaggcttgatggagaattttggttgggcctagagaagatatactccatagtgaagcaatctaattatgttttacgaattgagttggaagactggaaagacaacaaacattatattgaatattctttttacttgggaaatcacgaaaccaactatacgctacatctagttgcgattactggcaatgtccccaatgcaatcccggaaaacaaagatttggtgttttctacttgggatcacaaagcaaaaggacacttcaactgtccagagggttattcaggaggctggtggtggcatgatgagtgtggagaaaacaacctaaatggtaaatataacaaaccaagagcaaaatctaagccagagaggagaagaggattatcttggaagtctcaaaatggaaggttatactctataaaatcaaccaaaatgttgatccatccaacagattcagaaagctttgaatgagctagtgcggatccacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 32 - DNA, 24 bp, synthetic construct
  source 1..24: mol_type = other DNA
gaggaatcagatgaggatatggga

SEQ ID NO: 33 - DNA, 20 bp, synthetic construct
  source 1..20: mol_type = other DNA
aagcaggctgacttggttgc

SEQ ID NO: 34 - DNA, 26 bp, synthetic construct
  source 1..26: mol_type = other DNA
tcggtctcttcgactaatcccgccaa

SEQ ID NO: 35 - RNA, 21 nt, synthetic construct
  source 1..21: mol_type = other RNA
  modified_base 1: mod_base = OTHER; note = Nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide with phosphorothioate linkage
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  modified_base 20: mod_base = OTHER; note = 2'-fluoro modified nucleotide with phosphorothioate linkage
  modified_base 21: mod_base = OTHER; note = 2'-O-methyl modified nucleotide
tactgatcaaatatgttgagc

(19) United States
(12) Patent Application Publication
(10) Pub. No.: US 2026/0092279 A1
(43) Pub. Date: Apr. 2, 2026

(54) DOUBLE-STRANDED RNAI AGENTS AND COMPOSITIONS FOR REDUCING EXPRESSION OF ANGIOPOIETIN-LIKE 3 (ANGPTL3) AND METHODS OF USE THEREOF

(71) Applicants: ASTRAZENECA AB, Södertälje (SE); SILENCE THERAPEUTICS GMBH, Berlin (DE)

(72) Inventors: Ariana FOINQUINOS, Södertälje (SE); Eva HURT-CAMEJO, Södertälje (SE); Xiao-Rong PENG, Södertälje (SE); Eliot MORRISON, Berlin (DE); Stavros GIANNAKOPOULOS, Berlin (DE); Sibylle DAMES, Berlin (DE); Timo JOHANNSSEN, Berlin (DE); Lucas BETHGE, Berlin (DE); Steffen SCHUBERT, Berlin (DE); Marie WIKSTRÖM LINDHOLM, Berlin (DE)

(21) Appl. No.: 19/344,040

(22) Filed: Sep. 29, 2025

Related U.S. Application Data
(63) Continuation of application No. PCT/IB2025/051304, filed on Feb. 7, 2025.

(30) Foreign Application Priority Data
Feb. 9, 2024 (EP) 24156795.7

(51) Int. Cl. C12N 15/113; A61P 3/06

(52) U.S. Cl. CPC C12N 15/1136; A61P 3/06; C12N 2310/14; C12N 2310/315; C12N 2310/333; C12N 2310/3341; C12N 2310/335; C12N 2310/336; C12N 2310/351

(57) ABSTRACT

There are disclosed certain double-stranded RNA (dsRNA) or siRNA agents for inhibition of angiopoietin-like 3 gene expression, compositions that include angiopoietin-like 3 agents and methods of use thereof. The double-stranded RNA (dsRNA) or siRNA agents are particularly useful in the treatment or prophylaxis of cardiovascular disease and metabolic conditions as well as in the treatment of heart failure and chronic kidney disease.

### TECHNICAL FIELD

The technical field relates to certain double-stranded RNA (dsRNA) or siRNA agents for inhibition of angiopoietin-like 3 gene expression, compositions that include angiopoietin-like 3 agents and methods of use thereof.

### CROSS-REFERENCE TO RELATED APPLICATIONS

The present application is a continuation of International Patent Application No. PCT/IB2025/051304, filed Feb. 7, 2025, which claims the priority benefit of EP24156795.7, filed Feb. 9, 2024, each of which are hereby incorporated by reference in their entirety.

### SEQUENCE LISTING

The present application is being filed along with a Sequence Listing in electronic format. The Sequence Listing is provided as a file entitled Sequence_Listing_2943_3230001.xml, created on Sep. 26, 2025, which is 194,050 bytes in size. The information in the electronic format of the sequence listing is incorporated herein by reference in its entirety. Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, unless otherwise stated, including, but not limited to such nucleic acids having modified nucleobases.

### BACKGROUND

Angiopoietin-like 3 (also called ANGPTL3, ANGPL3, ANG3, or angiopoietin-like protein 3) is an angiopoietin protein encoded by the human angiopoietin-like 3 gene that is reported to be involved in regulating lipid metabolism. ANGPTL3 is a 460-amino acid polypeptide that consists of a signal peptide, N-terminal coiled-coil domain, and a C-terminal fibrinogen (FBN)-like domain. ANGPTL3 is known to be primarily produced in hepatocytes in humans, and after synthesis is secreted into circulation. ANGPTL3 acts as an inhibitor of lipoprotein lipase, which catalyzes hydrolysis of triglycerides, and endothelial lipase, which hydrolyzes lipoprotein phospholipids. Inhibition of these enzymes can cause increases in plasma levels of triglycerides, high-density lipoproteins (HDL), and phospholipids. Further, loss-of-function mutations in ANGPTL3 lead to familial hypobetalipoproteinemia, which is characterized by low levels of triglycerides and low-density lipoprotein (LDL-C) in plasma. In humans, loss of-function in ANGPTL3 is also correlated with a decreased risk of atherosclerotic cardiovascular disease.

An effective therapeutic that targets ANGPTL3 has been reported to potentially provide a beneficial impact in the treatment (including prophylactic treatment) of cardiometabolic diseases such as hypertriglyceridemia, obesity, hyperlipidemia, abnormal lipid and/or cholesterol metabolism, atherosclerosis type I diabetes mellitus. cardiovascular disease, coronary artery disease, non alcoholic steatohepatitis, non-alcoholic fatty liver disease, homozygous and heterozygous familial hypercholesterolemia, statin resistant hypercholesterolemia and other metabolic related disorders and diseases.

Certain double-stranded RNA (dsRNA) or siRNA agents have been identified as being capable of inhibiting the expression of an ANGPTL3 gene (see e.g. WO 2012/17784, WO 2016/168286, WO 2016/154127, WO 2019/055633 and WO 2021/188795), the specific ANGPTL3 siRNA agent disclosed herein were not previously disclosed or known, and enable highly efficient ANGPTL3-specific inhibition of expression of an ANGPTL3 gene.

That an effective therapeutic targeting ANPTL3 could provide a beneficial impact in the treatment (including prophylactic treatment) of heart failure, including HFpEF and HFrEF, or chronic kidney disease (CKD) has never been depicted.

### SUMMARY

There exists a need for novel ANGPTL3-specific RNA interference (RNAi) agents, e.g. double-stranded RNA (dsRNA) or siRNA agents, that are able to inhibit the expression of an ANGPTL3 gene in vitro and/or in vivo using the ANGPTL3 siRNA agents and compositions that include ANGPTL3 siRNA agents described herein. The ANGPTL3 siRNA agents described herein can selectively and efficiently decrease or inhibit expression of an ANGPTL3 gene, and thereby reducing triglyceride (TG) levels and/or cholesterol in a subject, e.g., a human or animal subject.

The described ANGPTL3 siRNA agents can be used in methods for therapeutic treatment (including prophylactic and preventive treatment) of symptoms and diseases associated with elevated TG levels and/or elevated cholesterol levels including but not limited to hypertriglyceridemia, obesity, hyperlipidemia. abnormal lipid and/or cholesterol metabolism, atherosclerosis type I diabetes mellitus. cardiovascular disease, coronary artery disease, non-alcoholic steatohepatitis, non-alcoholic fatty liver disease, homozygous and heterozygous familial hypercholesterolemia, statin resistant hypercholesterolemia and other metabolic related disorders and diseases. The ANGPTL3 siRNA agents disclosed herein can selectively reduce ANGPTL3 gene expression, which can lead to a reduction in, among other things, TG levels and or cholesterol levels, in a subject. The methods disclosed herein include the administration of one or more ANGPTL3 siRNA agents to a subject, e.g. a human or animal subject, using any suitable methods known in the art, such as subcutaneous injection or intravenous administration.

An object is to provide a method of treating symptoms and diseases associated with elevated TG levels and/or elevated cholesterol levels including but not limited to hypertriglyceridemia, obesity, hyperlipidemia. abnormal lipid and/or cholesterol metabolism, atherosclerosis type I diabetes mellitus. cardiovascular disease, coronary artery disease, non-alcoholic steatohepatitis, non-alcoholic fatty liver disease, homozygous and heterozygous familial hypercholesterolemia, statin resistant hypercholesterolemia and other metabolic related disorders and diseases comprising administering to a subject a therapeutically effective amount of a double-stranded siRNA comprising an antisense strand comprising a nucleobase sequence that is complementary to an equal length portion of a ANGPTL3 nucleic acid having the nucleobase sequence of SEQ ID NO: 1, and a sense strand, thereby treating hypertriglyceridemia, obesity, hyperlipidemia. abnormal lipid and/or cholesterol metabolism, atherosclerosis type I diabetes mellitus. cardiovascular disease, coronary artery disease, non-alcoholic steatohepatitis, non-alcoholic fatty liver disease, homozygous and heterozygous familial hypercholesterolemia, statin resistant hypercholesterolemia and other metabolic related disorders and diseases.

An object is to provide a method of treating heart failure comprising administering to a subject a therapeutically effective amount of a double-stranded siRNA comprising an antisense strand comprising a nucleobase sequence that is complementary to an equal length portion of a ANGPTL3 nucleic acid having the nucleobase sequence of SEQ ID NO: 1, and a sense strand, thereby treating heart failure.

A further object is to provide a method of treating chronic kidney disease comprising administering to a subject a therapeutically effective amount of a double-stranded siRNA comprising an antisense strand comprising a nucleobase sequence that is complementary to an equal length portion of a ANGPTL3 nucleic acid having the nucleobase sequence of SEQ ID NO: 1, and a sense strand, thereby treating chronic kidney disease.

In one embodiment, the ANGPTL3 siRNA agent is Conjugate 1, a double-stranded RNA (dsRNA) or siRNA capable of inhibiting expression of the human ANGPTL3 gene comprising (i) a first (sense) strand having the sequence:

(SEQ ID NO: 10)
NAG-invAb (ps) mG mC mU mC mA mA mC mA fU fA fU
mU mU mG mA mU mC mA mG mU mA (ps) invAb,

and (ii) a second (antisense) strand having the sequence:

(SEQ ID NO: 3)
mU (ps) fA (ps) mC (ps) fU mG fA mU fC mA fA mA
fU mA fU mG fU mU fG mA fG (ps) mC,

where NAG is a triantennary ligand moiety linked to the 5′ end of the first strand and having the structure:

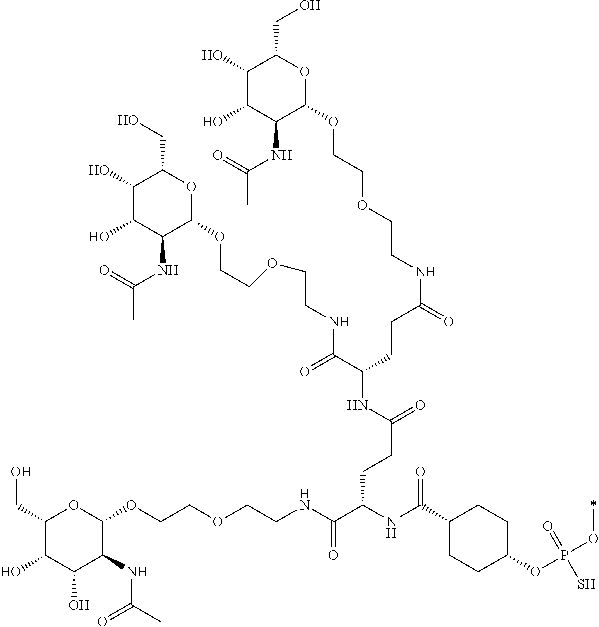 [SMILES: *OP(=O)(S)O[C@H]1CC[C@@H](C(=O)N[C@@H](CCC(=O)N[C@@H](CCC(=O)NCCOCCO[C@H]2O[C@@H](CO)[C@@H](O)[C@@H](O)[C@@H]2NC(C)=O)C(=O)NCCOCCO[C@H]2O[C@@H](CO)[C@@H](O)[C@@H](O)[C@@H]2NC(C)=O)C(=O)NCCOCCO[C@H]2O[C@@H](CO)[C@@H](O)[C@@H](O)[C@@H]2NC(C)=O)CC1]

In one embodiment, the ANGPTL3 siRNA agent is Conjugate 2, a double-stranded RNA (dsRNA) or siRNA capable of inhibiting expression of the human ANGPTL3 gene comprising (i) a first (sense) strand having the sequence:

(SEQ ID NO: 11)
[M]-mC mU mA mC mA mU fA fU fA mA mA mC mU mA mC
mA mA (ps) mG (ps) mU,

and (ii) a second (antisense) strand having the sequence:

(SEQ ID NO: 5)
mA (ps) fC (ps) mU fU mG fU mA fG mU fU mU fA mU
fA mU fG mU (ps) fA (ps) mG,

where [M] is a triantennary ligand moiety linked to the 5′ end of the first strand and having the structure:

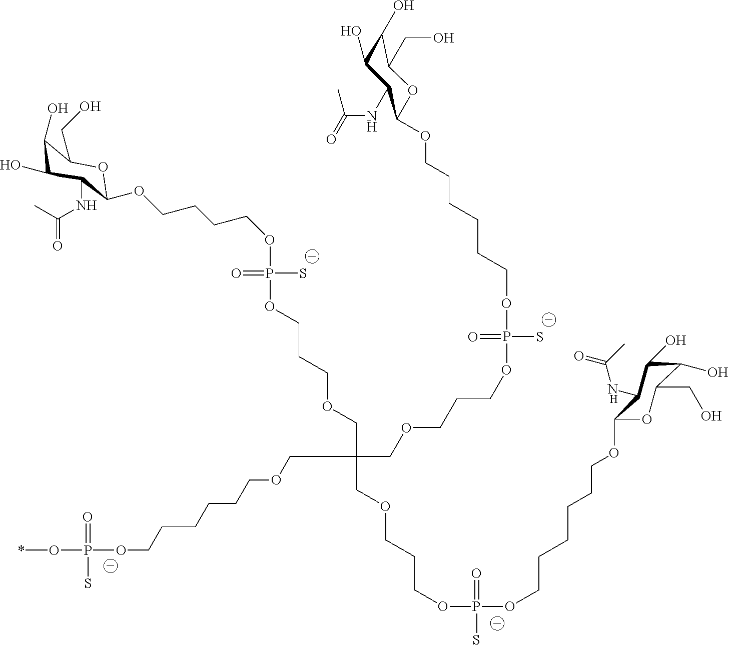

Thus, the terminal phosphorothioate group of the ligand moiety [M] is bonded directly (via the “free” bond indicated “*”) to the 5′ position of the 5′ terminal nucleotide of the first strand (i.e., to the 5′-carbon of the 2′-O-methyl ribose moiety of the mC residue at the 5′ end of the first strand). Sequences are shown in the conventional 5′ to 3′ direction.

In the context of the above formula, the following abbreviations are used:

| Abbreviation | Meaning |
|---|---|
| mA, mU, mC, mG | 2′-O-methyl ribonucleotide (adenine, uracil, cytosine, guanine) |
| fA, fU, fC, fG | 2′-fluoro-ribonucleotide (adenine, uracil, cytosine, guanine) |
| (ps) | phosphorothioate linkage between adjacent nucleotides |

In a further embodiment there is also provided a chirally enriched population of double-stranded siRNA, wherein the population is enriched for double-stranded siRNA comprising at least one particular phosphorothioate internucleoside linkage having a particular stereochemical configuration, or polynucleotide conjugates comprising said double-stranded siRNA.

The chirally enriched population may be enriched for double-stranded siRNA comprising at least one particular phosphorothioate internucleoside linkage having the (Sp) or (Rp) configuration, or polynucleotide conjugates comprising said double-stranded siRNA. The chirally enriched population may be enriched for double-stranded siRNA having a particular, independently selected stereochemical configuration at each phosphorothioate internucleoside linkage, or polynucleotide conjugates comprising said double-stranded siRNA. The chirally enriched population may be enriched for double-stranded siRNA having the (Rp) configuration at one particular phosphorothioate internucleoside linkage and the (Sp) configuration at each of the remaining phosphorothioate internucleoside linkages, or polynucleotide conjugates comprising said double-stranded siRNA. The chirally enriched population may be enriched for double-stranded siRNA having at least 2 or at least 3 contiguous phosphorothioate internucleoside linkages in the Sp, Sp, and Rp configurations, in the 5′ to 3′ direction, or polynucleotide conjugates comprising said double-stranded siRNA.

In a population of double-stranded siRNA comprising double-stranded or polynucleotide conjugates of the invention, all of the phosphorothioate internucleoside linkages of the double-stranded siRNA may be stereorandom.

In a further embodiment there is also provided a pharmaceutical composition comprising a double-stranded siRNA, polynucleotide conjugate, and a pharmaceutically acceptable diluent or carrier.

The pharmaceutically acceptable diluent may be water or phosphate-buffered saline. The pharmaceutical composition may consist essentially of said double-stranded siRNA, said polynucleotide conjugate and water or phosphate-buffered saline.

In a further embodiment there is also provided a method comprising administering to a subject said double-stranded siRNA, said polynucleotide conjugate, or said pharmaceutical composition.

In one embodiment, there is provided a method of treating a disease associated with ANGPTL3 comprising administering to a subject having a disease associated with ANGPTL3 a therapeutically effective amount of Conjugate 2 or said pharmaceutical composition, thereby treating the disease associated with ANGPTL3.

The disease associated with ANGPTL3 may be hypertriglyceridemia, obesity, hyperlipidemia, abnormal lipid and/or cholesterol metabolism, atherosclerosis, type 2 diabetes mellitus, cardiovascular disease, coronary artery disease, non-alcoholic steatohepatitis, non-alcoholic fatty liver disease, homozygous and heterozygous familial hypercholesterolemia, statin resistant hypercholesterolemia and other metabolic-related disorders and diseases.

In a further embodiment, there is provided a method of treating heart failure comprising administering to a subject a therapeutically effective amount of a double-stranded siRNA comprising an antisense strand comprising a nucleobase sequence that is at least 80% complementary to an equal length portion of a ANGPTL3 nucleic acid having the nucleobase sequence of SEQ ID NO: 1, and a sense strand, thereby treating heart failure.

In still a further embodiment, there is provided a method of treating heart failure comprising administering to a subject a therapeutically effective amount of a double-stranded siRNA comprising an antisense strand comprising a nucleobase sequence that is at least 85% complementary to an equal length portion of a ANGPTL3 nucleic acid having the nucleobase sequence of SEQ ID NO: 1, and a sense strand, thereby treating heart failure.

In still a further embodiment, there is provided a method of treating heart failure comprising administering to a subject a therapeutically effective amount of a double-stranded siRNA comprising an antisense strand comprising a nucleobase sequence that is at least 90% complementary to an equal length portion of a ANGPTL3 nucleic acid having the nucleobase sequence of SEQ ID NO: 1, and a sense strand, thereby treating heart failure.

In still a further embodiment, there is provided a method of treating heart failure comprising administering to a subject a therapeutically effective amount of a double-stranded siRNA comprising an antisense strand comprising a nucleobase sequence that is at least 95% complementary to an equal length portion of a ANGPTL3 nucleic acid having the nucleobase sequence of SEQ ID NO: 1, and a sense strand, thereby treating heart failure.

In still a further embodiment, there is provided a method of treating heart failure comprising administering to a subject a therapeutically effective amount of a double-stranded siRNA comprising an antisense strand comprising a nucleobase sequence that is 100% complementary to an equal length portion of a ANGPTL3 nucleic acid having the nucleobase sequence of SEQ ID NO: 1, and a sense strand; thereby treating heart failure.

In still a further embodiment, there is provided a method of treating heart failure comprising administering to a subject a therapeutically effective amount of Conjugate 2 or said pharmaceutical composition.

In still a further embodiment, there is provided a method of treating heart failure comprising administering to a subject a therapeutically effective amount of Conjugate 1 or said pharmaceutical composition.

In a further embodiment, there is provided a method of treating chronic kidney disease (CKD) comprising administering to a subject a therapeutically effective amount of a double-stranded siRNA comprising an antisense strand comprising a nucleobase sequence that is at least 80% complementary to an equal length portion of a ANGPTL3 nucleic acid having the nucleobase sequence of SEQ ID NO: 1, and a sense strand, thereby treating CKD.

In still a further embodiment, there is provided a method of treating chronic kidney disease (CKD) comprising administering to a subject a therapeutically effective amount of a double-stranded siRNA comprising an antisense strand comprising a nucleobase sequence that is at least 85% complementary to an equal length portion of a ANGPTL3 nucleic acid having the nucleobase sequence of SEQ ID NO: 1, and a sense strand, thereby treating CKD.

In still a further embodiment, there is provided a method of treating chronic kidney disease (CKD) comprising administering to a subject a therapeutically effective amount of a double-stranded siRNA comprising an antisense strand comprising a nucleobase sequence that is at least 90% complementary to an equal length portion of a ANGPTL3 nucleic acid having the nucleobase sequence of SEQ ID NO: 1, and a sense strand, thereby treating CKD.

In still a further embodiment, there is provided a method of treating chronic kidney disease (CKD) comprising administering to a subject a therapeutically effective amount of a double-stranded siRNA comprising an antisense strand comprising a nucleobase sequence that is at least 95% complementary to an equal length portion of a ANGPTL3 nucleic acid having the nucleobase sequence of SEQ ID NO: 1, and a sense strand, thereby treating CKD.

In still a further embodiment, there is provided a method of treating chronic kidney disease (CKD) comprising administering to a subject a therapeutically effective amount of a double-stranded siRNA comprising an antisense strand comprising a nucleobase sequence that is 100% complementary to an equal length portion of a ANGPTL3 nucleic acid having the nucleobase sequence of SEQ ID NO: 1, and a sense strand, thereby treating CKD.

In still a further embodiment, there is provided a method of treating chronic kidney disease (CKD) comprising administering to a subject a therapeutically effective amount of Conjugate 2 or said pharmaceutical composition.

In still a further embodiment, there is provided a method of treating chronic kidney disease (CKD) comprising administering to a subject a therapeutically effective amount of Conjugate 1 or said pharmaceutical composition.

### BRIEF DESCRIPTION OF THE DRAWINGS

FIG. 1 Five-concentration testing of ANGPTL3-targeting siRNAs Conjugate 2 and Conjugate 1 in primary human hepatocytes.

FIG. 2 Five-concentration testing of ANGPTL3-targeting siRNAs Conjugate 2 and Conjugate 1 in primary cynomolgus monkey hepatocytes.

FIG. 3 Baseline-corrected circulating human ANGPTL3 protein humanized mice treated with a single dose of conjugate 2.

FIG. 4 Human ANGPTL3 mRNA expression in liver on day 49 in humanized mice treated with a single dose of Conjugate 2.

FIG. 5 Human ANGPTL3 mRNA expression in liver on day 49 in humanized mice treated with a single dose of Conjugate 3 and 4, respectively.

FIG. 6 Schematic study outline Example 5, Therapeutic effects of Conjugate 1 in an obese HFpEF ZSF1 rat model In Vivo.

FIG. 7 Body weight (gr) at the end of the 12 weeks treatment period, Example 5.

FIG. 8 ANGPTL3 protein and TG plasma levels after administration of Conjugate 1 in ZSF-1 rats, Example 5.

FIG. 9a Heart content (lipidometic analysis) after administration of Conjugate 1 in ZSF-1 rats.

FIG. 9b Liver content (lipidometic analysis) after administration of Conjugate 1 in ZSF-1 rats.

FIG. 10 Diastolic function (IVRT and NT-proBNP) after Conjugate 1 administration in ZSF-1 rats.

FIG. 11 Tissue proteomics after Conjugate 1 administration in ZSF-1 rats.

FIG. 12 Renal health represented by UACR, podocyte health and glomerulosclerosis after Conjugate 1 administration in ZSF-1 Rats.

FIG. 13 Baseline and Endpoint Proton-Density Fat Fraction (PDFF) results in HfpEF NHPs after treatment with ANGPTL3 inhibition.

FIG. 14 Baseline and Endpoint Extracellular volume (ECV) results in HfpEF NHPs after treatment with ANGPTL3 inhibition.

FIG. 15 Left atria volume index (LAVi) results in HfpEF NHPs after treatment with ANGPTL3 inhibition.

FIG. 16 Plasma ANGPTL3 levels in HfpEF NHPs after treatment with ANGPTL3 inhibition.

FIG. 17 Plasma TG levels in HFpEF NHPs after treatment with ANGPTL3 inhibition.

### DETAILED DESCRIPTION

This detailed description and its specific examples, while indicating embodiments, are intended for purposes of illustration only. Therefore, there is no limitation to the illustrative embodiments described in this specification. In addition, it is to be appreciated that various features that are, for clarity reasons, described in the context of separate embodiments, also may be combined to form a single embodiment. Conversely, various features that are, for brevity reasons, described in the context of a single embodiment, also may be combined to form sub-combinations thereof. Listed below are definitions of various terms used in the specification and claims.

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

Unless specific definitions are provided, the nomenclature used in connection with, and the procedures and techniques of, analytical chemistry, synthetic organic chemistry, and medicinal and pharmaceutical chemistry described herein are those well-known and commonly used in the art.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts described herein to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

The polynucleotides of the present invention may be prepared by any means known in the art. For example, large amounts of the polynucleotides may be produced by replication in a suitable host cell. The polynucleotides of the present invention may preferably be produced by chemical synthesis, e.g., by the phosphoramidite method or the tri-ester method and may be performed on commercial automated oligonucleotide synthesizers. A double-stranded oligonucleotide may be obtained from the single stranded product of chemical synthesis either by synthesizing the complementary strand and annealing the strand together under appropriate conditions or by adding the complementary strand using DNA polymerase with an appropriate primer sequence.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

The terms “decrease” “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e., abrogation) as compared to a reference level.

The terms “increased”, “increase”, “enhance”, or “activate” are all used herein to mean an increase by a statically significant amount. The terms “increased”, “increase”, “enhance”, or “activate” can mean an increase of at least 25%, at least 50% as compared to a reference level, for example an increase of at least about 50%, or at least about 75%, or at least about 80%, or at least about 90%, at least about 95%, or at least about 98%, or at least about 99%, or at least about 100%, or at least about 250% or more compared with a reference level, or at least about a 1.5-fold, or at least about a 2-fold, or at least about a 2.5-fold, or at least about a 3-fold, or at least about a 4-fold, or at least about a 5-fold or at least about a 10-fold increase, or any increase between 1.5-fold and 10-fold or greater as compared to a reference level.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are oligomeric compounds comprising modified oligonucleotides.

As used herein, “complementary” in reference to an oligonucleotide means that is at least 70% of the nucleobases of the oligonucleotide and the nucleobases of another nucleic acid or one or more regions thereof are capable of hydrogen bonding with one another when the nucleobase sequence of the oligonucleotide and the other nucleic acid are aligned in opposing directions. “Complementary region” in reference to a region of an oligonucleotide means that is at least 70% of the nucleobases of that region and the nucleobases of another nucleic acid or one or more regions thereof are capable of hydrogen bonding with one another when the nucleobase sequence of the oligonucleotide and the other nucleic acid are aligned in opposing directions, i.e. the nucleobase sequences are reverse complementary. For the avoidance of doubt, all references herein to an oligonucleotide (e.g. an antisense strand of a double-stranded oligonucleotide of the invention) as complementary to a target sequence means that the nucleobase sequence of said oligonucleotide (e.g. the antisense strand of a double-stranded oligonucleotide of the invention) is reverse complementary to said target sequence.

Complementary nucleobases mean nucleobases that are capable of forming hydrogen bonds with one another. Complementary nucleobase pairs include adenine (A) and thymine (T), adenine (A) and uracil (U), cytosine (C) and guanine (G), 5-methyl cytosine (mC) and guanine (G). Certain modified nucleobases that pair with natural nucleobases or with other modified nucleobases are known in the art and are not considered complementary nucleobases as defined herein unless indicated otherwise. For example, inosine can pair, but is not considered complementary, with adenosine, cytosine, or uracil. Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that modifies one or more properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

As used herein, “internucleoside linkage” is the covalent linkage between adjacent nucleosides in an oligonucleotide. As used herein “modified internucleoside linkage” means any internucleoside linkage other than a phosphodiester internucleoside linkage.

As used herein, “motif” means the pattern of unmodified and/or modified sugar moieties, nucleobases, and/or internucleoside linkages, in an oligonucleotide.

As used herein, “modified nucleoside” means a nucleoside comprising a modified nucleobase and/or a modified sugar moiety.

As used herein, “nucleobase” means an unmodified nucleobase or a modified nucleobase. A nucleobase is a heterocyclic moiety. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one other nucleobase. A “5-methyl cytosine” is a modified nucleobase. A universal base is a modified nucleobase that can pair with any one of the five unmodified nucleobases.

As used herein, “nucleobase sequence” means the order of contiguous nucleobases in a nucleic acid or oligonucleotide independent of any sugar or internucleoside linkage modification. By way of example and without limitation, an oligomeric compound having the nucleobase sequence “ATCGATCG” encompasses any compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and compounds having other modified nucleobases, such as “AT^{m}CGAUCG,” wherein ^{m}C indicates a cytosine base comprising a methyl group at the 5-position. Finally, for clarity, unless otherwise indicated, the phrase “nucleobase sequence of SEQ ID NO: X”, refers only to the sequence of nucleobases in that SEQ ID NO.: X, independent of any sugar or internucleoside linkage modifications also described in such SEQ ID.

As used herein, “nucleoside” means a compound or fragment of a compound comprising a nucleobase and a sugar moiety. The nucleobase and sugar moiety are each, independently, unmodified or modified.

As used herein, “oligomeric agent” means a double-stranded oligonucleotide and optionally one or more additional features, such as a second oligonucleotide.

As used herein, “polynucleotide conjugate” means a double-stranded oligonucleotide and optionally one or more additional features, such as a conjugate group or terminal group.

A double-stranded oligonucleotide comprises a first oligonucleotide strand that is paired with a second oligonucleotide strand that is complementary to the first oligonucleotide strand. A double-stranded oligonucleotide comprises a sense oligonucleotide or strand (also known in the art as a passenger oligonucleotide or passenger strand), and an antisense oligonucleotide or strand (also known in the art as a guide oligonucleotide or guide strand).

As used herein, “antisense activity” means any detectable and/or measurable change attributable to the hybridization of an antisense strand to its target nucleic acid. In certain embodiments, antisense activity is a decrease in the amount or expression of a target nucleic acid or protein encoded by such target nucleic acid compared to target nucleic acid levels or target protein levels in the absence of the antisense strand. In certain embodiments, antisense activity is the modulation of splicing of a target pre-mRNA.

As used herein, “antisense agent” means an antisense strand and optionally one or more additional features, such as a sense strand. Thus, a double-stranded oligonucleotide of the invention may be described as an antisense agent.

As used herein, “antisense compound” means an antisense strand and optionally one or more additional features, such as a conjugate group. An antisense agent may comprise an antisense compound and a sense strand.

As used herein, “sense compound” means a sense strand and optionally one or more additional features, such as a conjugate group.

As used herein, “antisense oligonucleotide” means an oligonucleotide, including the oligonucleotide portion of an antisense compound, that is capable of hybridizing to a target nucleic acid and is capable of at least one antisense activity. Antisense oligonucleotides include but are not limited to antisense RNAi oligonucleotides and antisense RNase H oligonucleotides. The double-stranded oligonucleotides of the invention are typically siRNA and so comprise an antisense RNAi oligonucleotide.

As used herein, “sense oligonucleotide” or “sense strand” means an oligonucleotide, including the oligonucleotide portion of a sense compound, that is capable of hybridizing to an antisense oligonucleotide.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “stabilized phosphate group” means a 5′-phosphate analog that is metabolically more stable than a 5′-phosphate as naturally occurs on DNA or RNA.

As used herein, “standard cell assay” means the assays described in the Examples and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the(S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “target nucleic acid” and “target RNA” mean a nucleic acid that an oligomeric compound is designed to affect. Target RNA means an RNA transcript and includes pre-mRNA and mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNAi (ssRNAi), and microRNA, including microRNA mimics. RNAi agents may comprise conjugate groups and/or terminal groups. In certain embodiments, an RNAi agent modulates the amount and/or activity, of a target nucleic acid. The term RNAi agent excludes antisense agents that act through RNase H.

As used herein, “RNase H agent” means an antisense agent that acts through RNase H to modulate a target nucleic acid and/or protein encoded by a target nucleic acid. In certain embodiments, RNase H agents are single-stranded. In certain embodiments, RNase H agents are double-stranded. RNase H compounds may comprise conjugate groups and/or terminal groups. In certain embodiments, an RNase H agent modulates the amount and/or activity of a target nucleic acid. The term RNase H agent excludes antisense agents that act principally through RISC/Ago2.

As used herein, “reducing” or “inhibiting” ANGPTL3 means reducing expression of ANGPTL3 RNA and/or protein levels in the presence of an oligomeric compound or oligomeric agent described herein compared to expression of ANGPTL3 RNA and/or protein levels in the absence of an oligomeric compound or oligomeric agent described herein.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto. For example, the term “pharmaceutically acceptable” may refer to salts, excipients, carriers, diluents, etc. approved by a regulatory agency of the Federal or a state government, or listed in the U.S. Pharmacopeia, European Pharmacopeia, or other generally recognized pharmacopeia.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein “prodrug” means a therapeutic agent in a first form outside the body that is converted to a second form within an animal or cells thereof. Typically, conversion of a prodrug within the animal is facilitated by the action of an enzymes (e.g., endogenous or viral enzyme) or chemicals present in cells or tissues and/or by physiologic conditions. In certain embodiments, the first form of the prodrug is less active than the second form.

As used herein, “treating” means improving a subject's disease or condition by administering an oligomeric agent or oligomeric compound described herein. In certain embodiments, treating a subject improves a symptom relative to the same symptom in the absence of the treatment. In certain embodiments, treatment reduces in the severity or frequency of a symptom, or delays the onset of a symptom, slows the progression of a symptom, or slows the severity or frequency of a symptom.

As used herein, “therapeutically effective amount” means an amount of a pharmaceutical agent or composition that has been observed to provide a therapeutic benefit to an animal. For example, a therapeutically effective amount may be observed to improve a symptom of a disease.

The terms “individual”, “subject”, and “patient”, are used interchangeably herein to refer to a mammalian subject for whom diagnosis, prognosis, disease monitoring, treatment, therapy, and/or therapy optimisation is desired. The mammal can be (without limitation) a human, non-human primate, mouse, rat, dog, cat, horse, or cow. In a preferred embodiment, the individual, subject, or patient is a human. An “individual” may be an adult, juvenile or infant. An “individual” may be male or female.

A “subject in need” of treatment for a particular condition can be an individual having that condition, diagnosed as having that condition, or at risk of developing that condition.

A subject can be one who has been previously diagnosed with or identified as suffering from or having a condition in need of treatment or one or more complications or symptoms related to such a condition, and optionally, have already undergone treatment for a condition as defined herein or the one or more complications or symptoms related to said condition. Alternatively, a subject can also be one who has not been previously diagnosed as having a condition as defined herein or one or more or symptoms or complications related to said condition. For example, a subject can be one who exhibits one or more risk factors for a condition, or one or more or symptoms or complications related to said condition or a subject who does not exhibit risk factors.

As used herein, the term “healthy individual” refers to an individual or group of individuals who are in a healthy state, e.g., individuals who have not shown any symptoms of the disease, have not been diagnosed with the disease and/or are not likely to develop the disease e.g., a thrombotic event. Preferably said healthy individual(s) is not on medication affecting haemostasis and has not been diagnosed with any other disease. The one or more healthy individuals may have a similar sex, age, and/or body mass index (BMI) as compared with the test individual. Application of standard statistical methods used in medicine permits determination of normal levels of expression in healthy individuals, and significant deviations from such normal levels.

Herein the terms “control” and “reference population” are used interchangeably.

There is provided Conjugate 2, comprising (i) a first (sense) strand having the sequence:

(SEQ ID NO: 11)
[M]-mC mU mA mC mA mU fA fU fA mA mA mC mU mA mC
mA mA (ps) mG (ps) mU,

and (ii) a second (antisense) strand having the sequence:

(SEQ ID NO: 5)
mA (ps) fC (ps) mU fU mG fU mA fG mU fU mU fA mU
fA mU fG mU (ps) fA (ps) mG,

where [M] is a triantennary ligand moiety linked to the 5′ end of the second strand and having the structure:

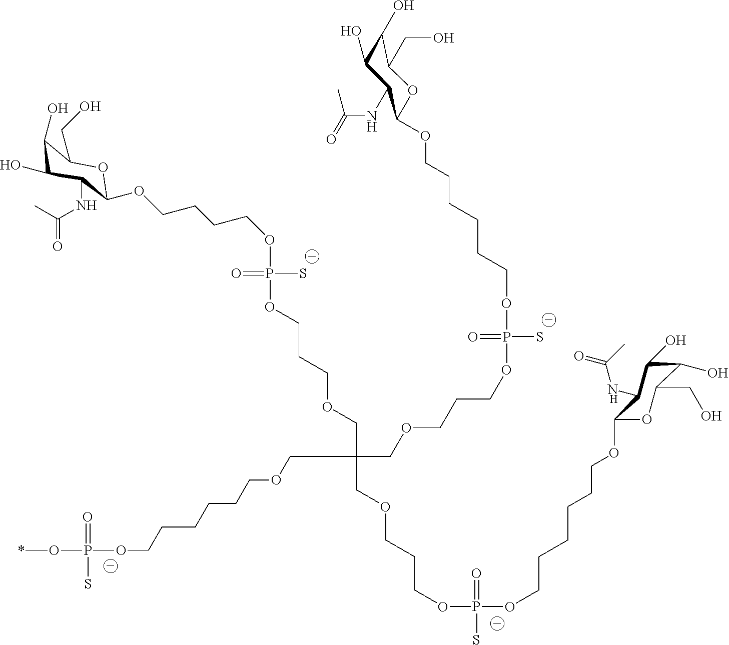

Thus, the terminal phosphorothioate group of the ligand moiety [M] is bonded directly (via the “free” bond indicated “*”) to the 5′ position of the 5′ terminal nucleotide of the first strand (i.e., to the 5′-carbon of the 2′-O-methyl ribose moiety of the mC residue at the 5′ end of the first strand).

Sequences are shown in the conventional 5′ to 3′ direction.

In the context of the above formula, the following abbreviations are used:

| Abbreviation | Meaning |
|---|---|
| mA, mU, mC, mG | 2′-O-methyl ribonucleotide (adenine, uracil, cytosine, guanine) |
| fA, fU, fC, fG | 2′-fluoro-ribonucleotide (adenine, uracil, cytosine, guanine) |
| (ps) | phosphorothioate linkage between adjacent nucleotides |

In one embodiment there is provided a process for the preparation of conjugate (I), or pharmaceutically acceptable salts of conjugate (I), and the intermediates used in the preparation thereof.

Another embodiment is a product obtainable by any of the processes or examples disclosed herein.

### Medical and Pharmaceutical Use

Conjugate 2, and pharmaceutically acceptable salts thereof, is believed to be useful in the prevention or treatment of cardiometabolic diseases such as hypertriglyceridemia, obesity, hyperlipidemia. abnormal lipid and/or cholesterol metabolism, atherosclerosis type I diabetes mellitus. cardiovascular disease, coronary artery disease, non alcoholic steatohepatitis, non-alcoholic fatty liver disease, homozygous and heterozygous familial hypercholesterolemia, statin resistant hypercholesterolemia and other metabolic related disorders and diseases in a mammal, particularly a human.

Conjugate 2, and pharmaceutically acceptable salts thereof, is believed to be useful in the prevention or treatment of heart failure in a mammal, particularly a human.

Conjugate 1, and pharmaceutically acceptable salts thereof, is believed to be useful in the prevention or treatment of heart failure in a mammal, particularly a human.

Conjugate 2, and pharmaceutically acceptable salts thereof, is believed to be useful in the prevention or treatment of chronic kidney disease in a mammal, particularly a human.

Conjugate 1, and pharmaceutically acceptable salts thereof, is believed to be useful in the prevention or treatment of chronic kidney disease in a mammal, particularly a human.

For the avoidance of doubt, as used herein, the term “treatment” includes therapeutic and/or prophylactic treatment.

When a compound or salt described herein is administered as therapy for treating a disorder, a “therapeutically effective amount” is an amount sufficient to reduce or completely alleviate symptoms or other detrimental effects of the disorder, cure the disorder, reverse, completely stop, or slow the progress of the disorder or reduce the risk of the disorder getting worse.

The compounds described herein are thus indicated both in the therapeutic and/or prophylactic treatment of these conditions.

The compounds described herein have the advantage that they may be more efficacious, be less toxic, be more selective, be more potent, produce fewer side effects, be more easily absorbed, and/or have a better pharmacokinetic profile (e.g. higher oral bioavailability and/or lower clearance), than compounds known in the prior art.

For the above-mentioned therapeutic indications, the dosage administered will vary with the compound employed, the mode of administration and the treatment desired. However, in general, satisfactory results are obtained when the compounds are administered at a dosage of the solid form of between 1 mg and 2000 mg per day.

Conjugate 2, and pharmaceutically acceptable salts thereof, may be used on their own, or in the form of appropriate pharmaceutical compositions in which the compound or derivative is in admixture with a pharmaceutically acceptable adjuvant, diluent or carrier. Thus, another aspect concerns a pharmaceutical composition comprising Conjugate 2, or a pharmaceutically acceptable salt thereof, in admixture with a pharmaceutically acceptable adjuvant, diluent or carrier.

Conjugate 1, and pharmaceutically acceptable salts thereof, may be used on their own, or in the form of appropriate pharmaceutical compositions in which the compound or derivative is in admixture with a pharmaceutically acceptable adjuvant, diluent or carrier. Thus, another aspect concerns a pharmaceutical composition comprising Conjugate 1, or a pharmaceutically acceptable salt thereof, in admixture with a pharmaceutically acceptable adjuvant, diluent or carrier.

In one embodiment there is provided Conjugate 2, or a pharmaceutically acceptable salt thereof for use in therapy, especially in the prevention or treatment of cardiometabolic diseases such as hypertriglyceridemia, obesity, hyperlipidemia. abnormal lipid and/or cholesterol metabolism, atherosclerosis type I diabetes mellitus. cardiovascular disease, coronary artery disease, non alcoholic steatohepatitis, non-alcoholic fatty liver disease, homozygous and heterozygous familial hypercholesterolemia, statin resistant hypercholesterolemia and other metabolic related disorders and diseases in a mammal, particularly a human.

In one embodiment there is provided Conjugate 2, or a pharmaceutically acceptable salt thereof for use in therapy, especially in the prevention or treatment of heart failure in a mammal, particularly a human.

In one embodiment there is provided Conjugate 1, or a pharmaceutically acceptable salt thereof for use in therapy, especially in the prevention or treatment of heart failure in a mammal, particularly a human.

In one embodiment there is provided Conjugate 2, or a pharmaceutically acceptable salt thereof for use in therapy, especially in the prevention or treatment of chronic kidney disease in a mammal, particularly a human.

In one embodiment there is provided Conjugate 1, or a pharmaceutically acceptable salt thereof for use in therapy, especially in the prevention or treatment of chronic kidney disease in a mammal, particularly a human.

These and other embodiments are described in greater detail herein below, where further aspects will be apparent to one skilled in the art from reading this specification.

### Combination Therapy

Conjugate 2, or pharmaceutically acceptable salts thereof, may also be administered in conjunction with other compounds used for the treatment of the above conditions.

In another embodiment, there is a combination therapy wherein Conjugate 2, or a pharmaceutically acceptable salt thereof, and a second active ingredient are administered concurrently, sequentially or in admixture, for the treatment of one or more of the conditions listed above. Such a combination may be used in combination with one or more further active ingredients.

When used in a combination therapy, it is contemplated that Conjugate 2, or pharmaceutically acceptable salts thereof, and the other active ingredients may be administered in a single composition, completely separate compositions, or a combination thereof. It also is contemplated that the active ingredients may be administered concurrently, simultaneously, sequentially, or separately. The particular composition(s) and dosing frequency(ies) of the combination therapy will depend on a variety of factors, including, for example, the route of administration, the condition being treated, the species of the patient, any potential interactions between the active ingredients when combined into a single composition, any interactions between the active ingredients when they are administered to the animal patient, and various other factors known to physicians (in the context of human patients), veterinarians (in the context of non-human patients), and others skilled in the art.

### Pharmaceutical Compositions

There is provided a method of treating a disease associated with ANGPTL3 comprising administering to a subject having a disease associated with ANGPTL3 a therapeutically effective amount of an ANPTL3 siRNA agent, thereby treating the disease associated with ANGPTL3.

Typically, the pharmaceutical composition further comprises a pharmaceutically acceptable diluent or carrier. The pharmaceutically acceptable diluent may be water or saline. A pharmaceutical composition may comprise or consist of a sterile saline solution and an ANPTL3 siRNA agent, which comprises an antisense strand having a nucleobase sequence complementary to a ANGPTL3 target nucleic acid. The sterile saline is preferably pharmaceutical grade saline.

A pharmaceutical composition may comprise or consist of an ANPTL3 siRNA agent, which comprises an antisense strand having a nucleobase sequence complementary to a ANGPTL3 target nucleic acid and sterile water. Preferably the sterile water is pharmaceutical grade water, e.g., water for injection. The saline may be phosphate-buffered saline (PBS), preferably sterile PBS.

A pharmaceutical composition may comprise an ANPTL3 siRNA agent, which comprises an antisense strand having a nucleobase sequence complementary to a ANGPTL3 target nucleic acid and one or more excipients. Excipients may be selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, an ANPTL3 siRNA agent, which comprises an antisense strand having a nucleobase sequence complementary to a ANGPTL3 target nucleic acid, may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

Pharmaceutical compositions comprising an ANGPTL3 siRNA agent, which comprises an antisense strand having a nucleobase sequence complementary to a ANGPTL3 target nucleic acid, encompass any pharmaceutically acceptable salts of the ANPTL3 siRNA agent. Pharmaceutical compositions comprising an ANPTL3 siRNA agent, which comprises an antisense strand having a nucleobase sequence complementary to a ANGPTL3 target nucleic acid, upon administration to an animal, including a human, are typically capable of providing (directly or indirectly) the biologically active metabolite or residue thereof. Accordingly, for example, the disclosure is also drawn to pharmaceutically acceptable salts of an ANGPTL3 siRNA agent, pharmaceutically acceptable salts of such prodrugs, and other bioequivalents. Suitable pharmaceutically acceptable salts include, but are not limited to, sodium and potassium salts.

A pharmaceutical composition may comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

A pharmaceutical composition may be prepared for administration by injection (e.g., intravenous, subcutaneous, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphate linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or a salt thereof” expressly includes all such forms that may be fully or partially protonated/de-protonated/in association with a cation. In certain instances, one or more specific cation is identified.

An ANGPTL3 siRNA agent, which comprises an antisense strand having a nucleobase sequence complementary to a ANGPTL3 target nucleic acid, may be in aqueous solution with sodium. An ANGPTL3 siRNA agent, which comprises an antisense strand having a nucleobase sequence complementary to a ANGPTL3 target nucleic acid, may be in aqueous solution with potassium. An ANGPTL3 siRNA agent, which comprises an antisense strand having a nucleobase sequence complementary to a ANGPTL3 target nucleic acid, may be in PBS. An ANGPTL3 siRNA agent, which comprises an antisense strand having a nucleobase sequence complementary to a ANGPTL3 target nucleic acid, may be in water. The pH of the solution may be adjusted with NaOH and/or HCl to achieve a desired pH.

### Preparation of the Compounds

### Example 1

Conjugate 1 (WO2019/055633: AD05488 (guide: AM07235-AS, passenger: AM07234-SS)):

Sense (passenger):
(SEQ ID NO: 10)
NAG-invAb (ps) mG mC mU mC mA mA mC mA fU fA fU
mU mU mG mA mU mC mA mG mU mA (ps) invAb
Antisense (guide):
(SEQ ID NO: 3)
mU (ps) fA (ps) mC (ps) fU mG fA mU fC mA fA mA
fU mA fU mG fU mU fG mA fG (ps) mC
SEQ ID NO: 2
invAb (ps) mG mC mU mC mA mA mC mA fU fA fU
mU mU mG mA mU mC mA mG mU mA (ps) invAb
SEQ ID NO: 3
mU (ps) fA (ps) mC (ps) fU mG fA mU fC mA fA mA
fU mA fU mG fU mU fG mA fG (ps) mC

### Single Strands Synthesis, Example 1

All synthetic reactions were performed under an inert atmosphere, unless otherwise stated. In the following examples, when the source of the starting products is not specified, it should be understood that said products are known compounds (e.g., commercially available compounds from suppliers such as Sigma-Aldrich) and/or may be prepared according to known methods, e.g. as described in the literature and patent publications described herein.

Nucleotide phosphoramidites were purchased from Sigma-Aldrich or WuXi. Linker phosphoramidites were purchased from Glen Research or WuXi. The 5′-amino-modifier C6 was obtained from GlenResearch. UV purities were determined using ion-pairing LCMS and are stated at 260 nm. Yields are given based on the initial resin loading and oligonucleotide content of the final product, as calculated from UV absorption.

### General Synthetic Procedure, Example 1

This procedure was used unless otherwise indicated.

Oligonucleotides SEQ ID NO: 2, 3 were synthesized on a 10 μmol scale on a K&A system using CUTAG CPG support (Sigma-Aldrich, 25-35 μmol/g).

Phosphoramidites were dissolved to a final concentration to 0.1 M (3 equivalents) in DNA grade acetonitrile (ACN) prior to use, except for the GalNAc phosphoramidite, dissolved at 0.2 M in dry DCM. Detritylation was performed using 3 vol-% dichloroacetic acid in DCM (contact time 5×35 s). Di- and tri-antennary linkers were deprotected using double detritylation. Activator BTT was used as activating agent (0.3 M in ACN) for the couplings.

Recirculation times of phosphoramidites were 10 min (single coupling) for all 2′-modified building blocks and 4*10 min (quadruple coupling) for GalNAc phosphoramidite (SEQ ID no 1). DDTT was dissolved in pyridine (0.2 M) and used as thiolation reagent with a contact time of 5 min. Oxidizer solution was purchased from Sigma-Aldrich and used as such with a contact time of 9 s. Equal volumes of Cap A (9.1 vol-% acetic anhydride in tetrahydrofuran (THF)) and Cap B (THF/N-methylimidazole/pyridine 80:10:10 vol-%) were mixed in situ for capping (contact time 50 s). Cyanoethyl backbone removal was performed with 20 vol-% diethylamine in ACN (contact time 7×1 min) after the synthesis complete. Oligonucleotides were cleaved from the solid support and further deprotected by treatment with ammonia (aq. 40%) solution (10 mL) at 55° C. for 18 h, and subsequently dried on a Speedvac.

### Purification Procedure, Example 1

The single strand oligonucleotides were purified using either ion-pairing HPLC or reverse phase HPLC.

Identity and purity of oligonucleotides was confirmed by liquid chromatography-mass spectrometry (LC-MS) using an Acquity I-class LC system, equipped with a PDA and coupled to an RDa via heated electrospray (BioAccord) by the Waters Corporation. Analytical runs were performed on an ACQUITY PREMIER BEH C18 1.7 μm 2.1×100 mm (130 Å) column at 80° C., using a gradient of 35-50% B (100% ACN) in A (10 mM TBAA in 10% ACN/90% H₂O) over 10 min (flow rate 0.5 mL/min).

Oligonucleotide SEQ ID NO 2 was purified using RP HPLC (XBridge C18, 5 μm 19×150 mm, A: 50 mM NH4HCO3 in water (pH8), B: ACN), gradient 5-12% B 16 min, 12-30% B 3 min, the repurified using same column and A: 60 mM DBuAA in (H2O/ACN 95/5) pH7.0, B: 60 mM DBuAA in ACN, gradient 10-40% in 12 min, 40-80% in 5 min, UV purity obtained is 95.0%; m/z (ESI−; TOF) calcd 2158.91 (4−); found: 2158.91.

Oligonucleotide SEQ ID NO 3 was purified using RP HPLC (XBridge C18, 5 μm 19×150 mm, A: 60 mM DBuAA in (H2O/ACN 95/5), pH 7.0, B: 60 mM DbuAA in ACN, gradient 20-50% in 12 min, 50-80% in 0.5 min, UV purity obtained is 90.2%; m/z (ESI−; TOF) calcd 1728.22 (4−); found: 1728.23.

Double Strands siRNA Synthesis-Annealing, Example 1

The amounts of the single strands were quantified by UV spectrophotometry at 260 nm and the solutions normalized to 2 mM using calculated extinction coefficients.

For siRNA compounds, single strands were mixed in equal molar amounts, warmed to 95° C. for 5 min and then left to cool down to room temperature over 1 h.

Double stranded siRNAs were analyzed using UPLC-MS method. Analyses of double stranded siRNAs were performed at ACQUITY PREMIER BEH Amide 1.7 μm 2.1×100 mm, 300 Å (30° C., flow 0.5 ml/min) using gradient: 15-60% B in 5 min, where A: 25 mM ammonium acetate in 70/30 ACN/water, B: 25 mM ammonium acetate in 30/70 ACN/water.

The final siRNAs were freeze-dried and redissolved in 1×PBS to a concentration of 10 mg/mL. Conjugate 1: Theoretical mass: 15564.6 Deconvolved mass found: 15564.7

### Example 2

### Conjugate 2:

Sense (passenger):
(SEQ ID NO: 11)
[M]-mC mU mA mC mA mU fA fU fA mA mA mC mU mA mC
mA mA (ps) mG (ps) mU
Antisense (guide):
(SEQ ID NO: 5)
mA (ps) fC (ps) mU fU mG fU mA fG mU fU mU fA mU
fA mU fG mU (ps) fA (ps) mG
SEQ ID NO: 4
mC mU mA mC mA mU fA fU fA mA mA mC mU mA mC mA
mA (ps) mG (ps) mU
SEQ ID NO: 5
mA (ps) fC (ps) mU fU mG fU mA fG mU fU mU fA mU
fA mU fG mU (ps) fA (ps) mG

### Building Block Synthesis, Example 2

Synthesis of the phosphoramidite derivatives of [M], (ST41-phos) as well as ST23 (ST23-phos) and their precursor compounds can be performed as described in WO2017/174657.

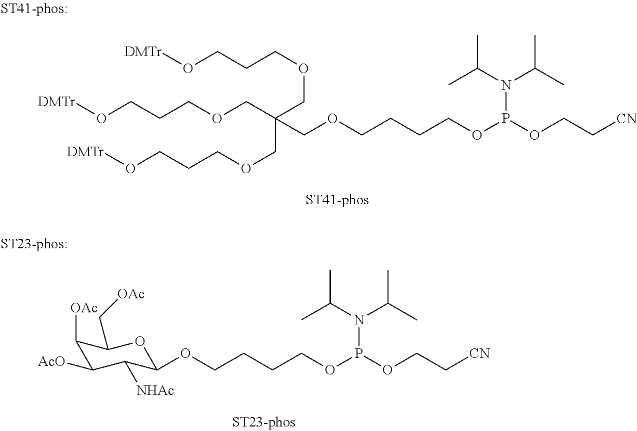

### Synthesis of Oligonucleotides, Example 2

Oligonucleotides were synthesized on an AKTA oligopilot 100 synthesizer using standard phosphoramidite chemistry. Commercially available base loaded solid support (Nitto phase HL250, Kinovate, CA), 2′OMe nucleotide phosphoramidites and 2′F nucleotide phosphoramidites (all standard protection. ChemGenes) were used according to the manufacturers recommended procedures.

Ancillary reagents were purchased from EMP Biotech. Synthesis was performed using a 0.1 M solution of the phosphoramidite in dry acetonitrile and benzylthiotetrazole (BTT) was used as activator (0.3M in acetonitrile). Coupling time was 10 min. A Cap/OX/Cap or Cap/Thio/Cap cycle was applied (Cap: Ac₂O/NMI/Lutidine/Acetonitrile. Oxidizer: 0.1M I₂ in pyridine/H₂O). Phosphorothioates were introduced using 0.2M XH (0.2 M Xanthane hydride in pyridine). DMT cleavage was achieved by treatment with 3% dichloroacetic acid in toluene. Upon completion of the programmed synthesis cycles a diethylamine (DEA) wash was performed. All other reagents and solvents were commercially available and used in standard reagent quality.

All oligonucleotides were synthesized in DMT-off mode. The single strands were cleaved off the support by 40% aq. methylamine treatment (90 min, RT). The resulting crude oligonucleotide was concentrated under reduced pressure to a concentration of ˜10 mg/mL and purified by ion exchange chromatography (SourceQ. 7.5 mL. GE Healthcare) on an AKTA Pure HPLC System using a sodium chloride gradient. Product containing fractions were pooled, desalted by UFDF (Sartorius Sartoflow Smart, Sartocon Slice 200 Hydrosart 2k, 10 times volume exchange against sterile water). To prove their purity all final single stranded products were analysed by AEX-HPLC (DNA Pac PA200 4.0×250 mm & DNAPac PA200 Guard 4×50 mm at 80° C., using a gradient of 25-70% B (10% MeCN, 20 mM TRIS, 0.4 M LiClO₄ in water, pH=7.4) in A (10% MeCN, 20 mM TRIS in water) over 10 min, flow rate 1 mL/min). Purity is given in % FLP (% full length product) which is the percentage of the UV-area under the assigned product signal in the UV-trace of the AEX-HPLC analysis of the final product. Identity of the respective single stranded products was confirmed by UPLC-MS analysis (Acquity UPLC BEH C18 2.1×50 mm, 1.7 μm Acquity BEH C18 1.7 μM Vanguard Pre-Col at 60° C., using a gradient of 10-22% B (100 mM HFIP, 15 mM TEA in MeOH) in A (100 mM HFIP, 15 mM TEA, 5% MeOH in water) over 5 min, flow rate 0.3 mL/min).

### Single Stranded Oligonucleotides:

| | MW. calc. | MW (ESI-), | % FLP |
|---|---|---|---|
| Name | (free acid) | found | (AEX-HPLC) |
| SEQ ID NO: 4 | 6247.85 Da | 6248.0 Da | 97.5 |
| SEQ ID NO: 5 | 7834.46 Da | 7835.0 Da | 94.9 |

### Double Strand Formation, Example 2

Individual single strands were dissolved in a concentration of 60 OD/mL in H₂O. Both individual oligonucleotide solutions were added together in a reaction vessel. A titration was performed for easier reaction monitoring. The first strand was added in 25% excess over the second strand as determined by UV-absorption at 260 nm. The reaction mixture was heated to 80° C. for 5 min and then slowly cooled to RT. Double strand formation was monitored by ion pairing reverse phase HPLC (X Bridge BEH C18 2.1×50 mm 2.5 μm, XP VanGuard Cartridges at 15° C., using a gradient of 5-45% B (100 mM HFIP, 15 mM TEA in MeOH) in A (100 mM HFIP, 15 mM TEA, 5% MeOH in water) over 15 min, flow rate 0.3 mL/min). From the UV-area of the residual single strand the needed amount of the second strand was calculated and added to the reaction mixture. The reaction was heated to 80° C. again and slowly cooled to RT. This procedure was repeated until less than 10% of residual single strand was detected.

### Nucleic Acid Conjugates:

| siRNA | % double strand |
|---|---|
| Conjugate 2 | 93.3 |

### Abbreviations Used in the Examples:

| Abbreviation | Meaning |
|---|---|
| mA, mU, mC, mG | 2′-O-methyl ribonucleotide (adenine, uracil, cytosine, guanine) |
| fA, fU, fC, fG | 2′-fluoro-ribonucleotide (adenine, uracil, cytosine, guanine) |
| (ps) | phosphorothioate linkage between adjacent nucleotides |
| invAb | abasic deoxyribose |
| T | Thymine |

[M] is:

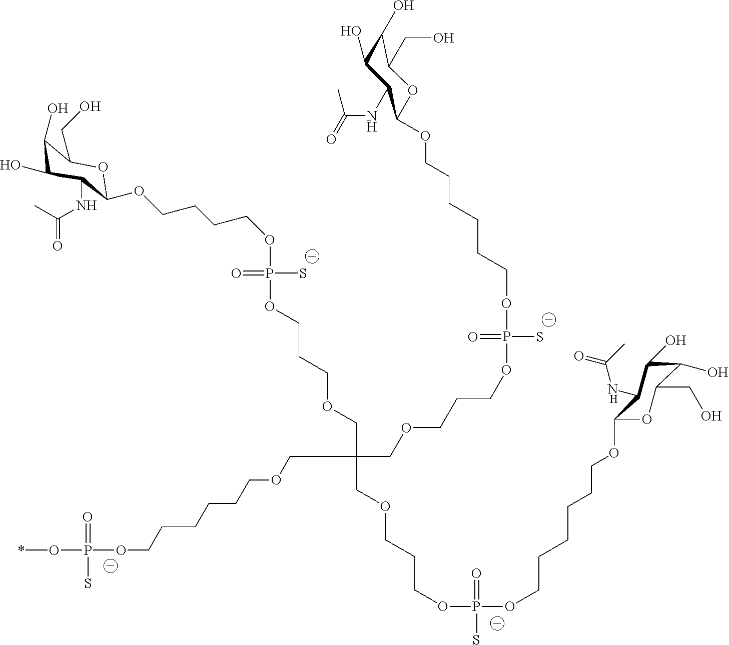

[M] is bonded via the “free” bond indicated “*”.

NAG is:

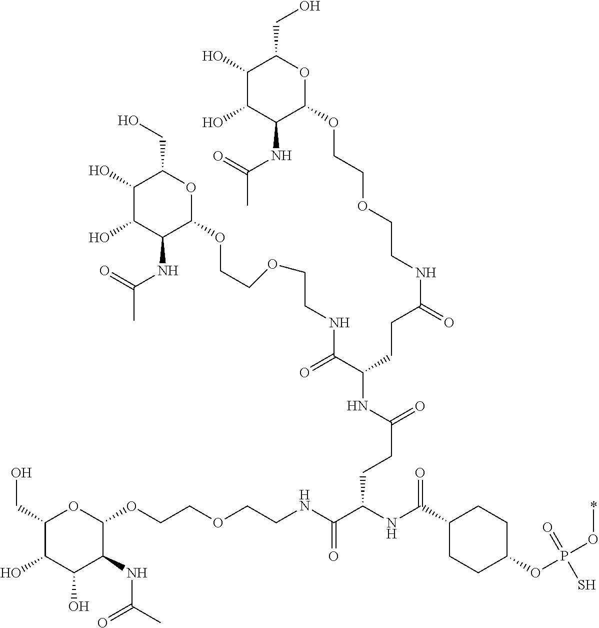 [SMILES: *OP(=O)(S)O[C@H]1CC[C@@H](C(=O)N[C@@H](CCC(=O)N[C@@H](CCC(=O)NCCOCCO[C@H]2O[C@@H](CO)[C@@H](O)[C@@H](O)[C@@H]2NC(C)=O)C(=O)NCCOCCO[C@H]2O[C@@H](CO)[C@@H](O)[C@@H](O)[C@@H]2NC(C)=O)C(=O)NCCOCCO[C@H]2O[C@@H](CO)[C@@H](O)[C@@H](O)[C@@H]2NC(C)=O)CC1]

NAG is bonded via the “free” bond indicated “*”.

### Example 3

In Vitro Test of GalNac-Conjugated siRNAs for Inhibition of ANGPTL3 mRNA Expression in Primary Hepatocytes

### List of Abbreviations for Example 3:

% Percent
AM Micromolar
A Adenine
ANGPTL3 Angiopoietin-like Protein 3
C Cytosine
c Sequence identity: cynomolgus
Ctr Non-targeting siRNA control
cyno Cynomolgus monkey
DMSO Dimethyl sulfoxide
G Guanine
GalNAc N-Acetylgalactosamine
h Sequence identity: human
LPR Lower primer for RT-PCR
mg Milligrams
ml Milliliter
nM Nanomolar
nt Nucleotides
PPIB Peptidyl-prolyl cis-trans isomerase B
PRB Fluorescently labelled probe for RT-PCR
RNA Ribonucleic acid
RNAi RNA interference
RT-qPCR Reverse transcription quantitative polymerase chain reaction
SD Standard deviation
siRNA Small interfering RNA
T Thymine
U Uracil
UPR Upper primer for PCR
UT Untreated
v/v Volume per volume

### Test Items

GalNAc-conjugated siRNAs tested were:

### Conjugate 1 (Example 1) and Conjugate 2 (Example 2).

### Cell Culture

Primary human hepatocytes (Lot Hu8264) and cynomolgus hepatocytes (crab-eating macaque, Macaca fascicularis, Lot CY411) were sourced from Life Technologies. As described by the manufacturer, primary hepatocytes were thawed and plated in Williams' E medium (Life Technologies), supplemented with 5% fetal bovine serum, 1 μM Dexamethasone in DMSO (final concentration of DMSO=0.01%) and 3.6% v/v of Thawing/Plating Cocktail-A (Thermo Fisher Scientific, CM3000) at 37° C. in a 5% CO₂ atmosphere.

Treatment of Primary Hepatocytes with GalNac-Conjugated siRNAs

Primary hepatocytes were seeded into collagen I-coated 96-well plates (Life Technologies) at a density of 35,000 cells and 45,000 cells per well for human and cynomolgus cells, respectively. GalNAc-conjugated siRNAs Conjugate 1 and Conjugate 2 were serially diluted to achieve final concentrations of 100, 20, 4, 0.8 and 0.16 nM for human cells, and 100, 10, 1, 0.1 and 0.01 nM for cynomolgus cells in the previously defined media in triplicates. Plates were then incubated at 37° C. in a 5% CO₂ atmosphere for 24 hours. Subsequently, cells were lysed, and RNA was isolated form the cells as described in section 4.4.

### Quantitative RT-PCR

Twenty-four hours post treatment with GalNAc conjugated siRNAs Conjugate 1 and Conjugate 2, total RNA was extracted using the InviTrap HTS 96-well kit (Invitek Molecular GmbH, Berlin, Germany) according to the manufacturer's instructions and eluted in 2×30 μl of elution buffer. Eluted RNA was stored at −80° C. until analysis by qPCR.

For qPCR, 10 μl of RNA were mixed with Takyon 5× Master Mix (Eurogentec, Seraing, Belgium) and primer/probe sets against either ANGPTL3 or PPIB (BioTez GmbH, Berlin, Germany or Eurogentec, Seraing, Belgium) as listed in Table 1:

TABLE 1
Sequences of primers and probes used for RT-qPCR.

| | Primer/ | SEQ | Sequence 5′ to 3′ |
|---|---|---|---|
| Nmae | Probe | ID NO: | |
| hANL3 | UPR | 12 | AGAGCACCAAGAACTACTCCCTTTC |
| | PRB | 13 | CATGATGGCATTCCTGCTGAATGTA |
| | | | CCA |
| | LWR | 14 | TGCCACTTGTATGTTCACCTCTGT |
| CANL3 | UPR | 12 | AGAGCACCAAGAACTACTCCCTTTC |
| | PRB | 13 | CATGATGGCATTCCTGCTGAATGTA |
| | | | CCA |
| | LWR | 15 | ACGTGCCACTTATATGTTCACCTCT |
| hcPPIB | UPR | 16 | AGATGTAGGCCGGGTGATCTTT |
| | PRB | 17 | TGTTCCAAAAACAGTGGATAATTTTG |
| | | | TGGCC |
| | LWR | 18 | GTAGCCAAATCCTTTCTCTCCTGT |

The RT-qPCR reactions were carried out at 20 ml per sample using a QuantStudio 6 Flex cycler (Applied Biosystems, part of Thermo Fisher Scientific, Massachusetts, USA) using standard protocols for RT-qPCR (48° C. 30 min, 95° C. 3 min, 40 cycles at 95° C. 15 s followed by 60° C. 1 min).

The data were calculated by using the comparative CT method also known as the 2^{-DDCt} method (Livak and Schmittgen, 2001 and Schmittgen and Livak, 2008). In brief, the amount of ANGPTL3 mRNA was normalized to the endogenous housekeeping gene PPIB followed by untreated control cells according to the following formula:

$$Fold - change = {2}^{- Δ Δ {C}_{T}} .$$

Data is presented as means of three replicates±SD.

### Results

Unless stated otherwise, all values presented in the text refer to mean±standard deviation (SD). Calculations of some tabular presentations were carried out using standard spreadsheet software (Graph Pad Prism 9, Microsoft® EXCEL®). A higher number of digits than tabulated were used for the calculation of some parameters. Therefore, recalculation of parameters from tabulated data may, in some instances, lead to minor variations. Since the original raw data is unaltered and thus numerically correct, this occurrence is not significant for the outcome of the study.

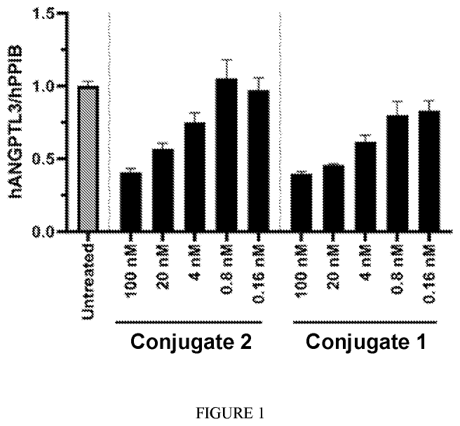

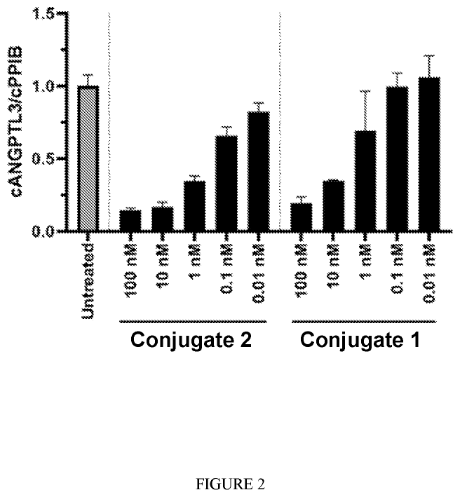

To estimate the impact of Conjugate 1 and Conjugate 2 on ANGPTL3 mRNA levels in vitro, primary human and cynomolgus hepatocytes were treated at five different concentrations for 24 hours, followed by isolation of RNA and quantification of remaining ANGPTL3 transcript levels by qPCR. Untreated cells were used for normalization. Experimental results are shown in FIG. 1 for human primary hepatocytes and in FIG. 2 for cynomolgus hepatocytes.

As illustrated in FIG. 1, Conjugate 2 induced a dose-dependent knockdown of ANGPTL3 in human hepatocytes, exceeding a 50% knockdown at 100 nM, whereas Conjugate 1 exceeded 50% at both 100 and 20 nM. The maximum knockdown measured in this experiment was 59±3% and 60.3±2% for Conjugate 2 and Conjugate 1, respectively.

In cynomolgus monkey hepatocytes (FIG. 2), tested compounds appeared more potent in general, which is in line with previous observations (data not shown), resulting in a 10-fold dilution series employed in this experiment. 50% knockdown levels were exceeded at 1 nM for Conjugate 2, and 10 nM for Conjugate 1. Maximum knockdown levels were observed at 100 nM in both cases (85.4±1% for Conjugate 2 vs. 80.4±1% for Conjugate 1).

Data is presented as means of three replicates±SD. Corresponding gene expression levels are depicted in Table 2.

TABLE 2
ANGPTL3 mRNA expression levels in primary
hepatocytes normalized to PPIB mRNA

| | Human hepatocytes | | | Cynomolgus hepatocytes | | |
|---|---|---|---|---|---|---|
| | siRNA | | | siRNA | | |
| siRNA | (conc.) | 2^{-DDCt} | SD 2^{-DDCt} | (conc.) | 2^{-DDCt} | SD 2^{-DDCt} |
| untreated | | 1.00 | 0.12 | | 1.00 | 0.17 |
| Conjugate 2 | 100 nM | 0.41 | 0.03 | 100 nM | 0.15 | 0.01 |
| | 20 nM | 0.57 | 0.04 | 10 nM | 0.16 | 0.03 |
| | 4 nM | 0.75 | 0.07 | 1 nM | 0.34 | 0.03 |
| | 0.8 nM | 1.05 | 0.13 | 0.1 nM | 0.66 | 0.06 |
| | 0.16 nM | 0.97 | 0.09 | 0.01 nM | 0.82 | 0.06 |
| Conjugate 1 | 100 nM | 0.40 | 0.02 | 100 nM | 0.22 | 0.01 |
| | 20 nM | 0.46 | 0.01 | 10 nM | 0.35 | 0.01 |
| | 4 nM | 0.62 | 0.05 | 1 nM | 0.65 | 0.27 |
| | 0.8 nM | 0.80 | 0.09 | 0.1 nM | 0.99 | 0.09 |
| | 0.16 nM | 0.83 | 0.07 | 0.01 nM | 0.97 | 0.02 |

### Reference Compounds Conjugate 3 and Conjugate 4

Conjugate 3 (WO2012/177784: AD-52988.1 (guide: A-108397.1, passenger A-108396.1):

Sense (passenger):
(SEQ ID NO: 19)
fU mA fC mA fU mA fU mA fA fA fC mU fA mC fA mA
fG mU fC mA fA-L96
Antisense (guide):
(SEQ ID NO: 7)
mU fU mG fA mC fU mU fG mU fA mG mU mU fU mA fU
mA fU mG fU mA (ps) fG (ps) mU
SEQ ID NO: 6
fU mA fC mA fU mA fU mA fA fA fC mU fA mC fA mA
fG mU fC mA fA
SEQ ID NO: 7
mU fU mG fA mC fU mU fG mU fA mG mU mU fU mA fU
mA fU mG fU mA (ps) fG (ps) mU

Conjugate 4 (WO2012/177784: AD-45887.1 (guide: A-96144.1, passenger: A-96143.1):

Sense (passenger):
(SEQ ID NO: 20)
mC mU fA mC fA mU fA mU fA fA fA mC mU fA mC fA
fA fG mU dT (ps) dT-L96
Antisense (guide):
(SEQ ID NO: 9)
fA fC fU fU fG mU fA fG fU fU mU fA mU fA fU fG
mU fA fG dT (ps) dT
SEQ ID NO: 8
mC mU fA mC fA mU fA mU fA fA fA mC mU fA mC fA
fA fG mU dT (ps) dT
SEQ ID NO: 9
fA fC fU fU fG mU fA fG fU fU mU fA mU fA fU fG
mU fA fG dT (ps) dT

In the context of the above formula, the following abbreviations are used:

| Abbreviation | Meaning |
|---|---|
| mA, mU, mC, mG | 2′-O-methyl ribonucleotide (adenine, uracil, cytosine, guanine) |
| fA, fU, fC, fG | 2′-fluoro-ribonucleotide (adenine, uracil, cytosine, guanine) |
| dT | 2′-deoxythymidine |
| (ps) | phosphorothioate linkage between adjacent nucleotides |

L96 is a triantennary ligand moiety having the structure:

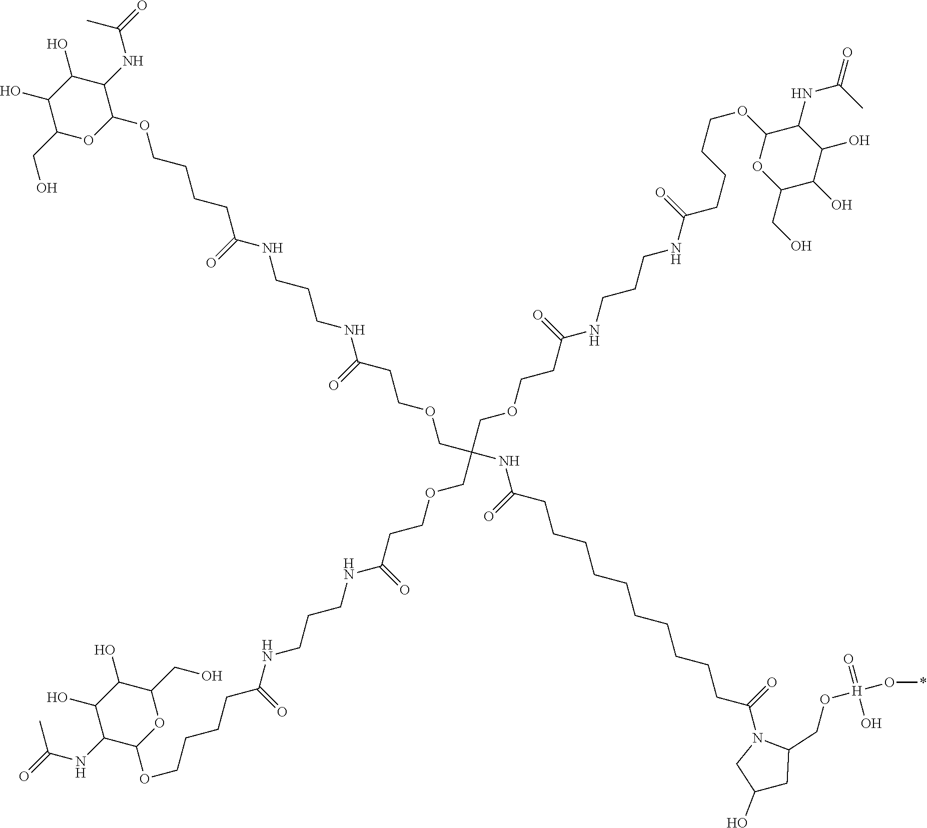

Thus, the terminal phosphorothioate group of the ligand moiety L96 is bonded directly (via the “free” bond indicated “*”) to the 3′ position of the 3′ terminal nucleotide of the first strand.

### Single Strands Synthesis

All synthetic reactions were performed under an inert atmosphere, unless otherwise stated. In the following examples, when the source of the starting products is not specified, it should be understood that said products are known compounds (e.g., commercially available compounds from suppliers such as Sigma-Aldrich) and/or may be prepared according to known methods, e.g. as described in the literature and patent publications described herein.

Nucleotide phosphoramidites were purchased from Sigma-Aldrich or WuXi. Linker phosphoramidites were purchased from Glen Research or WuXi. The 5′-amino-modifier C6 was obtained from GlenResearch. UV purities were determined using ion-pairing LCMS and are stated at 260 nm. Yields are given based on the initial resin loading and oligonucleotide content of the final product, as calculated from UV absorption.

### General Synthetic Procedure

This procedure was used unless otherwise indicated.

Oligonucleotides SEQ ID NO: 7, 9 were synthesised on an ÄKTA OligoPilot Plus 10 synthesizer (GE Healthcare), on a 32 μmole scale, using a standard synthesis cycle of detritylation (3% dichloroacetic acid in toluene), coupling (coupling agent: 0.25 M 5-[3,5-Bis(trifluoromethyl)phenyl]-1H-tetrazole solution in acetonitrile), capping (Cap A: 20% N-methylimidazole and 80% acetonitrile; Cap B: 20% pyridine, 20% acetic anhydride and 60% acetonitrile), oxidation (0.05 M iodine in pyridine and water) or thiolation (0.2 M Xanthane hydride in pyridine), and solid supports (UNY Primer Support 5G ˜353 μmole/g, GE Healthcare). All fully protected β-cyanoethyl phosphoramidite monomers were dissolved in anhydrous acetonitrile (0.1 M) under argon immediately prior to use. The phosphoramidite re-circulation time/coupling time for DNA monomers was 5 min and was extended to 10 min for RNA monomers. Stepwise coupling efficiencies and overall yields were determined by automated trityl cation absorption monitoring exceeding 98% for all oligonucleotides synthesized. At the end of solid phase synthesis, the solid-support bound oligonucleotides were treated with ammonia (aq. 40%) solution (10 mL) at 55° C. for 18 h, and subsequently dried on a Speedvac.

Oligonucleotides SEQ ID NO: 6, 8 were synthesised on an ÄKTA OligoPilot Plus 10 synthesizer (GE Healthcare) on a 13 μmole scale, using a standard synthesis cycle described above and solid support Nitto Phase (loading 0.153 mmol/g) preloaded with L96 GalNac.

### Purification Procedure

The single strand oligonucleotides were purified using either ion-pairing HPLC or reverse phase HPLC.

Identity and purity of oligonucleotides was confirmed by liquid chromatography-mass spectrometry (LC-MS) using an Acquity I-class LC system, equipped with a PDA and coupled to an RDa via heated electrospray (BioAccord) by the Waters Corporation. Analytical runs were performed on an ACQUITY PREMIER BEH C18 1.7 μm 2.1×100 mm (130 Å) column at 80° C., using a gradient of 35-50% B (100% ACN) in A (10 mM TBAA in 10% ACN/90% H₂O) over 10 min (flow rate 0.5 mL/min).

Oligonucleotide SEQ ID NO 6 was purified using RP HPLC (XBridge C18, 5 μm 19×150 mm, A: 50 mM NH4HCO3 in water (pH8), B: ACN), gradient 5% B for 1 min, 5-20% B in 11 min, 20-90% B in 3 min, UV purity obtained is 94%; m/z (ESI−; TOF) calcd 1717.19 (5−); found: 1718.0.

Oligonucleotide SEQ ID NO 7 was purified using IP HPLC (XBridge C18, 5 μm 19×150 mm, A: 60 mM DBuAA (H2O/ACN 95/5) pH7, B: 60 mM DBuAA in ACN), gradient 5% B for 1 min, 20-40% B in 12 min, 50-80% in 0.5 min, UV purity obtained is 95.6%; m/z (ESI−; TOF) calcd 1879.0 (4−); found: 1878.98.

Oligonucleotide SEQ ID NO 8 was purified using RP HPLC (XBridge C18, 5 μm 19×150 mm, A: 50 mM NH4HCO3 in water (pH8), B: ACN), gradient 5% B for 1 min, 5-18% B in 11 min, 18-90% B in 3 min, UV purity obtained is 96.5%; m/z (ESI−; TOF) calcd 1709.59 (5−); found: 1710.3.

Oligonucleotide SEQ ID NO 9 was purified using IP HPLC (XBridge C18, 5 μm 19×150 mm, A: 60 mM DBuAA (H2O/ACN 95/5) pH7, B: 60 mM DBuAA in ACN), gradient 5% B for 1 min, 20-40% B in 12 min, 40-80% in 0.5 min, UV purity obtained is 96.3%; m/z (ESI−; TOF) calcd 2243.27 (3−); found: 2243.26.

Double Strands siRNA Synthesis-Annealing

The amounts of the single strands were quantified by UV spectrophotometry at 260 nm and the solutions normalized to 2 mM using calculated extinction coefficients.

For siRNA compounds, single strands were mixed in equal molar amounts, warmed to 95 C for 5 min and then left to cool down to room temperature over 1 h.

Double stranded siRNAs were analyzed using UPLC-MS method. Analyses of double stranded siRNAs were performed at ACQUITY PREMIER BEH Amide 1.7 μm 2.1×100 mm, 300 Å (30° C., flow 0.5 ml/min) using gradient: 15-60% B in 5 min, where A: 25 mM ammonium acetate in 70/30 ACN/water, B: 25 mM ammonium acetate in 30/70 ACN/water.

The final siRNAs were freeze-dried and redissolved in 1×PBS to a concentration of 10 mg/mL.

Conjugate 3: Theoretical mass: 16118.8 Deconvolved mass found: 16118.6

Conjugate 4: Theoretical mass: 15293.2 Deconvolved mass found: 15293.1

### Example 4

### In Vivo ANGPTL3 Knockdown Using AAV-Based Humanized ANGPTL3 Model.

The aim of the study was to profile the in vivo effects of Conjugate 2 in its ability to knockdown (KD) human ANGPTL3 mRNA in the liver and reduction of circulating ANGPTL3 protein as compared to Conjugates 3 and 4.

### List of Abbreviations for Example 4:

AAT Alpha1-antitrypsin
AAV Associated Adeno Virus
ApoE Apolipoprotein E
EDTA Ethylendiaminetatraacetic acid
ELISA Enzyme-linked immunosorbent assay
siRNA Small interfering RNA
HDL High-density lipoprotein
KD Knockdown
LDL-C Low Density Lipoprotein-Cholesterol
LPL Lipoprotein lipase
ORF Open reading frame
PBS Phosphate-buffered saline
PCR Polymerase chain reaction
RNA Ribonucleic acid
UTR Untranslated region
VLDL Very low-density lipoprotein

### Test Formulation

Compounds: Conjugate 2
   Conjugate 3
   Conjugate 4
Vehicle: PBS
Stock concentration: 10 mg/mL
Dosing solution: 0.05, 0.2 and 0.6 mg/mL

### Test System In Vivo

Species: Mouse
Strain: C57Bl6N
Sex: Females
Total No of animals: 50
Body weight range: 21.0-26.3 grams at study start
Supplier: Charles River Laboratory, Germany
Identification method: Tail tattoo
Acclimatisation: At least 5 days
Housing conditions: Upon arrival, mice were group housed in Macrolon L3 cages. Twelve-hour light/dark cycle (lights on at 6 am), 40 to 60 humidity, 20 to 22° C.
Water: Bottled tap water, ad lib
Diet: Normal chow diet ad lib (R70, Lantmännen, Kimstad, Sweden)/(A40 Safe, Augy, France)
Bedding: Aspen wood chip bedding (Harlan, USA)
Pre-treatment: humanANGPTL3 AAV 5E+11gc
Participation in previous studies: No

### Study Design

Dose(s): 0.25, 1 or 3 mg/kg of Conjugate 2
   0.25, 1 or 3 mg/kg of Conjugate 3
   0.25, 1 or 3 mg/kg of Conjugate 4
Volume(s) of administration: 5 mL/kg
Route(s) and frequency of
Administration: S.c.
Number/group: 5

### In Vivo Design

Mice were 8 weeks of age when they arrived the test facility. All mice were put on regular chow diet from the day of arrival and throughout the study. After 6 weeks or 6 days of acclimatization, the mice were injected via the tail vein with AAV. Two weeks after the AAV injection each group was then treated, receiving the assigned treatment via a single subcutaneous injection. Blood samples from the tail vein collected in EDTA glass capillaries (REF #164113 Micro Haematocrit Tubes, Vitrex Medical) were taken on day 0, 7, 22, 30, 42 and 49. Termination was carried out after the last blood sample.

At termination, all animals were anesthetized with isoflurane (Abbot, Stockholm, Sweden) using a vaporizer (Univentor 400, Agnthos, Sweden) and blood was withdrawn from the orbital plexus and collected into tubes containing EDTA as anticoagulant (REF #15.1671.101, Multivette 600K3E, Sarstedt). Blood samples were centrifuged at 10.000× g for 4 minutes at 4° C. temperature. The resulting plasma was aliquoted and stored at −20° C. until further analysis was performed. Subsequently, anesthetized mice were culled by cutting out the heart. The left lateral lobe from the liver was collected and snap frozen in liquid nitrogen and stored at −80° C. until subsequent analysis.

### AAV-ANGPTL3 Generation

The liver-targeted AAV vector genome construct which carries a liver-specific hAAT-hApoE promoter (a chimeric promoter with human apolipoprotein E and alpha-1-antitrypsin gene enhancer/promoter) was previously reported ((Liu, 2023 #499). Human ANGPTL3 open reading frame (ORF) sequence was synthesized by GenScripts, and the ORF was amplified by PCR using a primer pair 5′-TTTGAATTCGCCACCATGTTCACAATTAAGCTCCTTCTT-3′ (SEQ ID NO: 21) and 5′-TACATTGCTAGCTCATTCAAAGCTTTCTGAATCTG-3′ (SEQ ID NO: 22). The PCR products were then purified using QIAquick PCR Purification Kit, digested with EcoRI and NheI and gel purified using QIAquick Gel Extraction Kit. The digested ANGPTL3 ORF was then cloned into the EcoRI and SpeI sites of AAV-hAAT-hApoE-FGF21 mutant vector plasmid, which led to the introduction of human ANGPTL3 ORF under the control of the liver specific hAAT-hApoE promoter. The resulting plasmid was named as pAAV-hAAT-hApoE-ANGPTL3_noUTRs and the insert was confirmed by sequencing using the following primers.

H83 ANGPTL3_L1
(SEQ ID NO: 23)
TCATGCCTCTTTGCACCATTC
H84 ANGPTL3_L2
(SEQ ID NO: 24)
AATGGCCTCCTTCAGTTGGG
H85 ANGPTL3_L3
(SEQ ID NO: 25)
GCCAAGAGCACCAAGAACTAC
H86 ANGPTL3_L4
(SEQ ID NO: 26)
CACTTCAACTGTCCAGAGGGT
H87 ANGPTL3_R1
(SEQ ID NO: 27)
TGTCTTGATCAATTCTGGAGGA
H88 ANGPTL3_R2
(SEQ ID NO: 28)
ATTGGTCTTCCACGGTCTGG
H89 ANGPTL3_R3
(SEQ ID NO: 29)
TGTTTGTTGTCTTTCCAGTCTTCC
H90 ANGPTL3_R4
(SEQ ID NO: 30)
CCTTGCTCCATACCACCCC

The full sequence of pAAV-hAAT-hApoE-ANGPTL3_noUTRs is as follows.

```
pAAV-hAAT-hApoE-ANGPTL3_noUTRs 6628 bp DNA
FEATURES Location/Qualifiers
Annotation 143 . . . 172
                  /label = “Annotation Promoter lac_promoter”
misc feature 237 . . . 377
                /label = “AAV ITR”
misc_feature 446 . . . 1167
                /label = “hAAT-hApoE prom”
misc_feature 1203 . . . 1695
                /label = “Human beta globin intron”
CDS 1714 . . . 3096
                /note = “ANGPTL3 CDS”-highlighted in bold
Terminator 3111 . . . 3589
                /label = “Terminator human_growth_hormone_poly_A”
Reporter 3749 . . . 3909
                /label = “Reporter lacZ_alpha_fragment”
Promoter 4753 . . . 4781
                /label = “Promoter ampicillin_resistance_gene(b-lactamase)promoter”
Selection 4823 . . . 5683
                /label = “Selection ampicillin_resistance_gene_ORF
Origin 5838 . . . 64567
                /label = “Origin pBR322_replication_origin
ORIGIN
(SEQ ID NO: 31
      1 agcgcccaat acgcaaaccg cctctccccg cgcgttggcc gattcattaa tgcagctggc
    61 acgacaggtt tcccgactgg aaagcgggca gtgagcgcaa cgcaattaat gtgagttagc
  121 tcactcatta ggcaccccag gctttacact ttatgcttcc ggctcgtatg ttgtgtggaa
  181 ttgtgagcgg ataacaattt cacacaggaa acagctatga ccatgattac gaattgcctg
  241 caggcagctg cgcgctcgct cgctcactga ggccgcccgg gcaaagcccg ggcgtcgggc
  301 gacctttggt cgcccggcct cagtgagcga gcgagcgcgc agagagggag tggccaactc
  361 catcactagg ggttcctatc gatatcaagc tttaatagta atcaattacg gggtcattag
  421 ttcatagccc atatatggag ttccgaggct cagaggcaca caggagtttc tgggctcacc
  481 ctgccccctt ccaacccctc agttcccatc ctccagcagc tgtttgtgtg ctgcctctga
  541 agtccacact gaacaaactt cagcctactc atgtccctaa aatgggcaaa cattgcaagc
  601 agcaaacagc aaacacacag ccctccctgc ctgctgacct tggagctggg gcagaggtca
  661 gagacctctc tgggcccatg ccacctccaa catccactcg accccttgga atttcggtgg
  721 agaggagcag aggttgtcct ggcgtggttt aggtagtgtg agaggggtac ccggggatct
  781 tgctaccagt ggaacagcca ctaaggattc tgcagtgaga gcagagggcc agctaagtgg
  841 tactctccca gagactgtct gactcacgcc accccctcca ccttggacac aggacgctgt
  901 ggtttctgag ccaggtacaa tgactccttt cggtaagtgc agtggaagct gtacactgcc
  961 caggcaaagc gtccgggcag cgtaggcggg cgactcagat cccagccagt ggacttagcc
1021 cctgtttgct cctccgataa ctggggtgac cttggttaat attcaccagc agcctccccc
1081 gttgcccctc tggatccact gcttaaatac ggacgaggac agggccctgt ctcctcagct
1141 tcaggcacca ccactgacct gggacagtgg tttagtggat atccttaagg gcccagccgg
1201 cccgaatccc ggccgggaac ggtgcattgg aacgcggatt ccccgtgcca agagtgacgt
1261 aagtaccgcc tatagagtct ataggcccac aaaaaatgct ttcttctttt aatatacttt
1321 tttgtttatc ttatttctaa tactttccct aatctctttc tttcagggca ataatgatac
1381 aatgtatcat gcctctttgc accattctaa agaataacag tgataatttc tgggttaagg
1441 caatagcaat atttctgcat ataaatattt ctgcatataa attgtaactg atgtaagagg
1501 tttcatattg ctaatagcag ctacaatcca gctaccattc tgcttttatt ttatggttgg
1561 gataaggctg gattattctg agtccaagct aggccctttt gctaatcatg ttcatacctc
1621 ttatcttcct cccacagctc ctgggcaacg tgctggtctg tgtgctggcc catcactttg
1681 gcaaagaatt gggattcgcg agaattcgcc accatgttca caattaagct ccttcttttt
1741 attgttcctc tagttatttc ctccagaatt gatcaagaca attcatcatt tgattctcta
1801 tctccagagc caaaatcaag atttgctatg ttagacgatg taaaaatttt agccaatggc
1861 ctccttcagt tgggacatgg tcttaaagac tttgtccata agacgaaggg ccaaattaat
1921 gacatatttc aaaaactcaa catatttgat cagtcttttt atgatctatc gctgcaaacc
1981 agtgaaatca aagaagaaga aaaggaactg agaagaacta catataaact acaagtcaaa
2041 aatgaagagg taaagaatat gtcacttgaa ctcaactcaa aacttgaaag cctcctagaa
2101 gaaaaaattc tacttcaaca aaaagtgaaa tatttagaag agcaactaac taacttaatt
2161 caaaatcaac ctgaaactcc agaacaccca gaagtaactt cacttaaaac ttttgtagaa
2221 aaacaagata atagcatcaa agaccttctc cagaccgtgg aagaccaata taaacaatta
2281 aaccaacagc atagtcaaat asaagaaata gaaaatcagc tcagaaggac tagtattcaa
2341 gaacccacag aaatttctct atcttccaag ccaagagcac caagaactac tccctttctt
2401 cagttgaatg aaataagaaa tgtaaaacat gatggcattc ctgctgaatg taccaccatt
2461 tataacagag gtgaacatac aagtggcatg tatgccatca gacccagcaa ctctcaagtt
2521 tttcatgtct actgtgatgt tatatcaggt agtccatgga cattaattca acatcgaata
2581 gatggatcac aaaacttcaa tgaaacgtgg gagaactaca aatatggttt tgggaggctt
2641 gatggagaat tttggttggg cctagagaag atatactcca tagtgaagca atctaattat
2701 gttttacgaa ttgagttgga agactggaaa gacaacaaac attatattga atattctttt
2761 tacttcggaa atcacgaaac caactatacg ctacatctag ttgcgattac tggcaatgtc
2821 cccaatgcaa tcccggaaaa caaagatttg gtgttttcta cttgggatca caaagcaaaa
2881 ggacacttca actgtccaga gggttattca ggaggctggt ggtggcatga tgagtgtgga
2941 gaaaacaacc taaatggtaa atataacaaa ccaagagcaa aatctaagcc agagaggaga
3001 agaggattat cttggaagtc tcaaaatgga aggttatact ctataaaatc aaccaaaatg
3061 ttgatccatc caacagattc agaaagcttt gaatgagcta gtgcggatcc acgggtggca
3121 tccctgtgac ccctccccag tgcctctcct ggccctggaa gttgccactc cagtgcccac
3181 cagccttgtc ctaataaaat taagttgcat cattttgtct gactaggtgt ccttctataa
3241 tattatgggg tggagggggg tggtatggag caaggggcaa gttgggaaga caacctgtag
3301 ggcctgcggg gtctattggg aaccaagctg gagtgcagtg gcacaatctt ggctcactgc
3361 aatctccgcc tcctgggttc aagcgattct cctgcctcag cctcccgagt tgttgggatt
3421 ccaggcatgc atgaccaggc tcagctaatt tttgtttttt tggtagagac ggggtttcac
3481 catattggcc aggctggtct ccaactccta atctcaggtg atctacccac cttggcctcc
3541 caaattgctg ggattacagg cgtgaaccac tgctcccttc cctgtcctta tcgatagatc
3601 taggaacccc tagtgatgga gttggccact ccctctctgc gcgctcgctc gctcactgag
3661 gccgggcgac caaaggtcgc ccgacgcccg ggctttgccc gggcggcctc agtgagcgag
3721 cgagcgcgca gctgcctgca ggcagcttgg cactggccgt cgttttacaa cgtcgtgact
3781 gggaaaaccc tygcgttacc caacttaatc gccttgcagc acatccccct ttcgccagct
3841 ggcgtaatag cgaagaggcc cgcaccgatc gcccttccca acagttgcgc agcctgaatg
3901 gcgaatggcg cctgatgcgg tattttctcc ttacgcatct gtgcggtatt tcacaccgca
3961 tacgtcaaag caaccatagt acgcgccctg tagcggcgca ttaagcgcgg cgggtgtggt
4021 ggttacgcgc agcgtgaccg ctacacttgc cagcgcccta gcgcccgctc ctttcgcttt
4081 cttcccttcc tttctcgcca cgttcgccgg ctttccccgt caagctctaa atcgggggct
4141 ccctttaggg ttccgattta gtgctttacg gcacctcgac cccaaaaaac ttgatttggg
4201 tgatggttca cgtagtgggc catcgccctg atagacggtt tttcgccctt tgacgttgga
4261 gtccacgttc tttaatagtg gactcttgtt ccaaactgga acaacactca accctatctc
4321 gggctattct tttgatttat aagggatttt gccgatttcg gcctattggt taaaaaatga
4381 gctgatttaa caaaaattta acgcgaattt taacaaaata ttaacgttta caattttatg
4441 gtgcactctc agtacaatct gctctgatgc cgcatagtta agccagcccc gacacccgcc
4501 aacacccgct gacgcgccct gacgggcttg tctgctcccg gcatccgctt acagacaagc
4561 tgtgaccgtc tccgggagct gcatgtgtca gaggttttca ccgtcatcac cgaaacgcgc
4621 gagacgaaag ggcctcgtga tacgcctatt tttataggtt aatgtcatga taataatggt
4681 ttcttagacg tcaggtggca cttttcgggg aaatgtgcgc ggaaccccta tttgtttatt
4741 tttctaaata cattcaaata tgtatccgct catgagacaa taaccctgat aaatgcttca
4801 ataatattga aaaaggaaga gtatgagtat tcaacatttc cgtgtcgccc ttattccctt
4861 ttttgcggca ttttgccttc ctgtttttgc tcacccagaa acgctggtga aagtaaaaga
4921 tgctgaagat cagttgggtg cacgagtggg ttacatcgaa ctggatctca acagcggtaa
4981 gatccttgag agttttcgcc ccgaagaacg ttttccaatg atgagcactt ttaaagttct
5041 gctatgtggc gcggtattat cccgtattga cgccgggcaa gagcaactcg gtcgccgcat
5101 acactattct cagaatgact tggttgagta ctcaccagtc acagaaaagc atcttacgga
5161 tggcatgaca gtaagagaat tatgcagtgc tgccataacc atgagtgata acactgcggc
5221 caacttactt ctgacaacga tcggaggacc gaaggagcta accgcttttt tgcacaacat
5281 gggggatcat gtaactcgcc ttgatcgttg ggaaccggag ctgaatgaag ccataccaaa
5341 cgacgagcgt gacaccacga tgcctgtagc aatggcaaca acgttgcgca aactattaac
5401 tggcgaacta cttactctag cttcccggca acaattaata gactggatgg aggcggataa
5461 agttgcagga ccacttctgc gctcggccct tccggctggc tggtttattg ctgataaatc
5521 tggagccggt gagcgtgggt ctcgcggtat cattgcagca ctggggccag atggtaagcc
5581 ctcccgtatc gtagttatct acacgacggg gagtcaggca actatggatg aacgaaatag
5641 acagatcgct gagataggtg cctcactgat taagcattgg taactgtcag accaagttta
5701 ctcatatata ctttagattg atttaaaact tcatttttaa tttaaaagga tctaggtgaa
5761 gatccttttt gataatctca tgaccaaaat cccttaacgt gagttttcgt tccactgagc
5821 gtcagacccc gtagaaaaga tcaaaggatc ttcttgagat cctttttttc tgcgcgtaat
5881 ctgctgcttg caaacaaaaa aaccaccgct accagcggtg gtttgtttgc cggatcaaga
5941 gctaccaact ctttttccga aggtaactgg cttcagcaga gcgcagatac caaatactgt
6001 tcttctagtg tagccgtagt taggccacca cttcaagaac tctgtagcac cgcctacata
6061 cctcgctctg ctaatcctgt taccagtggc tgctgccagt ggcgataagt cgtgtcttac
6121 cgggttggac tcaagacgat agttaccgga taaggcgcag cggtcgggct gaacgggggg
6181 ttcgtgcaca cagcccagct tggagcgaac gacctacacc gaactgagat acctacagcg
6241 tgagctatga gaaagcgcca cgcttcccga agggagaaag gcggacaggt atccggtaag
6301 cggcagggtc ggaacaggag agcgcacgag ggagcttcca gggggaaacg cctggtatct
6361 ttatagtcct gtcgggtttc gccacctctg acttgagcgt cgatttttgt gatgctcgtc
6421 aggggggcgg agcctatgga aaaacgccag caacgcggcc tttttacggt tcctggcctt
6481 ttgctggcct tttgctcaca tgttctttcc tgcgttatcc cctgattctg tggataaccg
6541 tattaccgcc tttgagtgag ctgataccgc tcgccgcagc cgaacgaccg agcgcagcga
6601 gtcagtgagc gaggaagcgg aag
```

Generation of recombinant AAV vectors: Ad293 cells (Agilent, Santa Clara, CA, USA) were plated in a five layered chamber in Gibco DMEM supplemented with 10% Gibco FBS and 1% Gibco penicillin-streptomycin (Thermo Fisher Scientific, Waltham, MA, USA). When these cells reached at 60-85% confluency under the microscope, they were transfected by polyethylenimine (Polyscience, Torrance, CA, USA) with triple plasmids, including pHelper containing adenoviral E2A and E4 genes, pRep2Cap8 encoding AAV2 Rep proteins and AAV8 serotype capsid, and pAAV-hAAT-hApoE-ANGPTL3_noUTRs, in a ratio of 2:1.4:1, respectively ((Liu, 2023 #499). After 72 hr of the post-transfection, cells were harvested and lysed via three freeze-thaw cycles followed by 1 hr of benzonase treatment at 37° C. Supernatants were further purified using iodixanol gradient-based ultracentrifugation. Titers of AAV vector preps used for in vivo study were quantitated by quantitative PCR. Since the recombinant AAV8 vector was generated by a standard and helper-free 3 plasmid transfection system, this vector does not express AAV8 and adenoviral helper proteins and cannot replicate in transduced hepatocytes.

Human ANGPTL3 mRNA Analysis

RNA was isolated from ˜20 mg snap-frozen liver using RNeasy Mini Kit (Qiagen, cat #74104) according to manufacturer's instructions. cDNA was generated using a High-Capacity RNA-to-CDNA™ Kit from Applied Biosystems (ThermoFisher cat #4387406) and used for real-time PCR with the QuantStudio 7 Flex (Applied Biosystems). ANGPTL3 gene expression was measured using TaqMan Gene Expression assays from Applied Biosystems (ThermoFisher, ANGPTL3: FAM-MGB Hs00205581 ml cat #4331182, and mouse RPLPO primer probe set from (Sigma Aldrich) as housekeeping gene.

Forward primer: 5′-GAGGAATCAGATGAGGATATGGGA-3′ (SEQ ID NO: 32),

Reverse primer 5′-AAGCAGGCTGACTTGGTTGC-3′ (SEQ ID NO: 33) and

FAM-TAM labelled probe 5′-TCGGTCTCTTCGACTAATCCCGCCAA-3′ (SEQ ID NO: 34) Real-time PCR was performed on a Quantstudio 7 flex and PCR cycle numbers (Ct) determined using the Quantstudio Real-Time PCR Software (Applied Biosystems). ANGTPL3 gene expression level was normalized against RPLPO the housekeeping gene.

Human ANGPTL3 mRNA Analysis

Human ANGPTL3 was measured in mouse plasma using Human Angiopoietin-like 3 Quantikine ELISA Kit (DANL30, R&D Systems, Minneapolis, MN). Prior to use, the ELISA assay was shown to not cross-react with endogenous mouse ANGPTL3 present in the plasma samples (BS003151-48). The mouse plasma samples were analysed for human ANGPTL3 according to the manufacture instructions with the following modifications; the calibration curve was extended with 4 points to cover 0.02-10 ng/mL, the plasma samples were diluted 25-, 50-, or 100-fold prior to analysis. Calibration standards were analysed in duplicated and study samples as single samples, the ELISA plates were read in an absorbance microplate reader capable of measuring absorbance at 450 nm, with correction wavelength set at 540 nm (e.g. iX3, Molecular Devices). The response of the standard curve was plotted as absorbance units on the linear scale versus concentration on the log scale and the 4-parameter function was used for curve fitting (using SoftMax Pro). To control for between plate variations a human quality control samples was run, in duplicate, in the beginning and end of each analysis plate. ANGPLT3 levels was reported as ng/mL plasma.

### Data Analysis

Plasma ANGPTL3 protein data were baseline-corrected, individually for each animal. The mean and standard error of each group at each day were calculated. Normality of data was investigated using the one-sample Kolmogorov-Smirnov test. Log-transformed data were suggested and used in subsequent tests. For human ANGPTL3 mRNA expression data, mean and standard error of each group were calculated. Normality of data was investigated using the one-sample Kolmogorov-Smirnov test. Log-transformed data were suggested and used in subsequent tests. For all analyses, Tukey's post hoc test was used for multiple comparisons. Calculations were performed in MATLAB (R2022b; MathWorks). Specifically, the function kstest was used to test for normality, and the functions anoval and multcompare were used for multiple comparison tests.

### Results

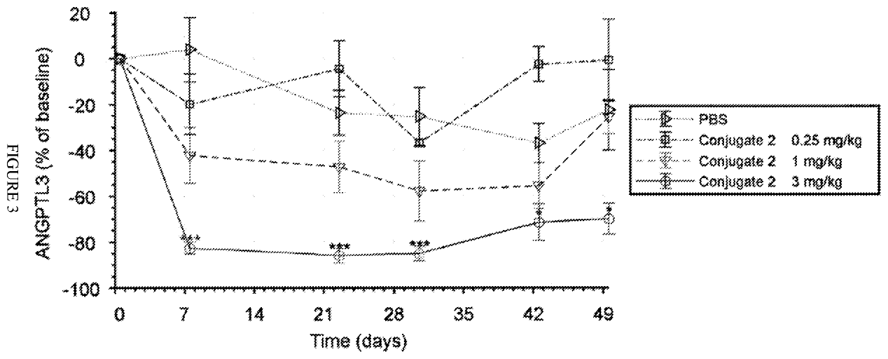

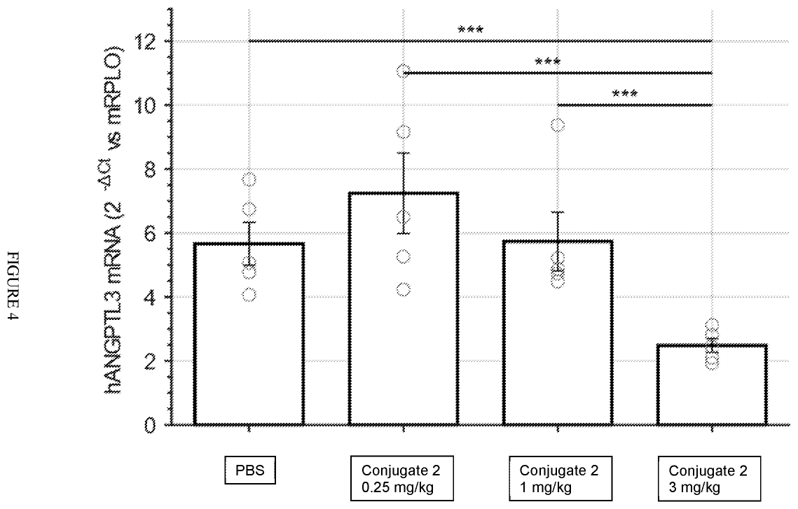

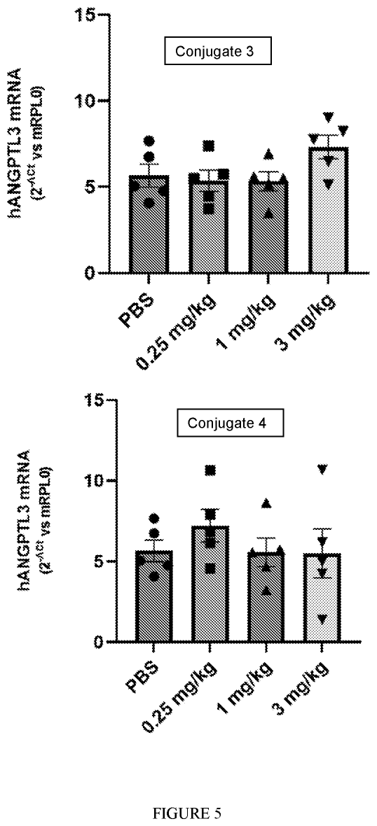

For Conjugate 2, maximum reductions of baseline-corrected ANGPTL3 protein concentrations were 37%, 58%, and 86% for single doses of 0.25, 1, and 3 mg/kg, respectively (Table 3; FIG. 3). For the treated groups, the maximum reductions were observed 22 to 30 days after dosing. The baseline-corrected ANGPTL3 protein concentration decreased over time in the placebo group with a maximum reduction of 37% at Day 42 and a reduction of 22% at the study end (Day 49). Neither the 0.25 mg/kg group nor the 1 mg/kg group showed statistically significant reductions in baseline-corrected ANGPTL3 protein concentration compared to placebo at the P<0.05 level. The high-dose group of 3 mg/kg showed statistically significant reduction in baseline-corrected ANGPTL3 protein concentration compared to placebo at all measured time-points (P<0.05). For the 1 mg/kg group, the baseline-corrected ANGPTL3 protein concentration started to return towards baseline, and reached the same level as the placebo group, but did not fully return to baseline, at Day 49. For the 3 mg/kg group, the baseline-corrected ANGPTL3 protein concentration did not fully return to baseline during the 7-week study. At study termination on Day 49, knockdown of human ANGPTL3 mRNA in liver was 56% compared to placebo (P<0.005 versus all other groups) for the high-dose group of 3 mg/kg. The groups dosed with 0.25 and 1 mg/kg did not show knockdown of human ANGPTL3 mRNA in the liver at Day 49 (Table 4; FIG. 4). For Conjugate 3, maximum reductions of baseline-corrected ANGPTL3 protein concentrations were 31%, 28%, and 43% for single doses of 0.25, 1, and 3 mg/kg, respectively (Table 3). For Conjugate 4, maximum reductions of baseline-corrected ANGPTL3 protein concentrations were 0%, 20%, and 30% for single doses of 0.25, 1, and 3 mg/kg, respectively (Table 3). None of the dose groups for Conjugates 3 or 4 did show any knockdown of human ANGPTL3 mRNA in the liver at Day 49 (Table 4; FIG. 5).

TABLE 3
Circulating Human ANGPTL3 Protein (% of baseline) In Humanized Mice Treated
with A Single Dose Of Conjugate 2, Conjugate 3 or Conjugate 4.

| | Dose | | | Day | Day | Day | Day | Day |
|---|---|---|---|---|---|---|---|---|
| Treatment | (mg/kg) | | Baseline | 7 | 22 | 30 | 42 | 49 |
| PBS | 0 | | 100 | 136 | 74 | 92 | 66 | 78 |
| PBS | 0 | | 100 | 66 | 45 | 44 | 32 | 40 |
| PBS | 0 | | 100 | 137 | 68 | 45 | 67 | 38 |
| PBS | 0 | | 100 | 90 | 97 | 105 | 66 | 127 |
| PBS | 0 | | 100 | 91 | 98 | 88 | 85 | 104 |
| | | N | 5 | 5 | 5 | 5 | 5 | 5 |
| | | Mean | 100 | 104 | 76 | 75 | 63 | 78 |
| Conjugate 2 | 0.25 | | 100 | 70 | 85 | 65 | 89 | 76 |
| Conjugate 2 | 0.25 | | 100 | 128 | 111 | 57 | 97 | 145 |
| Conjugate 2 | 0.25 | | 100 | 87 | 134 | 63 | 102 | 128 |
| Conjugate 2 | 0.25 | | 100 | 61 | 83 | 65 | 77 | 46 |
| Conjugate 2 | 0.25 | | 100 | 55 | 64 | 65 | 122 | 101 |
| | | N | 5 | 5 | 5 | 5 | 5 | 5 |
| | | Mean | 100 | 80 | 96 | 63 | 97 | 99 |
| Conjugate 2 | 1 | | 100 | 39 | 25 | 31 | 25 | 54 |
| Conjugate 2 | 1 | | 100 | 45 | 52 | 90 | 81 | 98 |
| Conjugate 2 | 1 | | 100 | 45 | 49 | 12 | 29 | 64 |
| Conjugate 2 | 1 | | 100 | 105 | 94 | 34 | 42 | 78 |
| Conjugate 2 | 1 | | 100 | 54 | 45 | 45 | 46 | 79 |
| | | N | 5 | 5 | 5 | 5 | 5 | 5 |
| | | Mean | 100 | 58 | 53 | 42 | 45 | 75 |
| Conjugate 2 | 3 | | 100 | 13 | 5 | 6 | 16 | 26 |
| Conjugate 2 | 3 | | 100 | 21 | 22 | 21 | 44 | 56 |
| Conjugate 2 | 3 | | 100 | 17 | 19 | 22 | 51 | 17 |
| Conjugate 2 | 3 | | 100 | 11 | 8 | 8 | 11 | 20 |
| Conjugate 2 | 3 | | 100 | 25 | 16 | 18 | 21 | 32 |
| | | N | 5 | 5 | 5 | 5 | 5 | 5 |
| | | Mean | 100 | 17 | 14 | 15 | 29 | 30 |
| Conjugate 3 | 0.25 | | 100 | 103 | 81 | 94 | 79 | 53 |
| Conjugate 3 | 0.25 | | 100 | 122 | 132 | 137 | 95 | 94 |
| Conjugate 3 | 0.25 | | 100 | 113 | 93 | 85 | 58 | 85 |
| Conjugate 3 | 0.25 | | 100 | 102 | 84 | 98 | 59 | 75 |
| Conjugate 3 | 0.25 | | 100 | 69 | 66 | 68 | 54 | 57 |
| | | N | 5 | 5 | 5 | 5 | 5 | 5 |
| | | Mean | 100 | 102 | 91 | 96 | 69 | 73 |
| Conjugate 3 | 1 | | 100 | 69 | 89 | 118 | 101 | 77 |
| Conjugate 3 | 1 | | 100 | 80 | 71 | 38 | 46 | 42 |
| Conjugate 3 | 1 | | 100 | 161 | 138 | 121 | 173 | 102 |
| Conjugate 3 | 1 | | 100 | 129 | 85 | 40 | 72 | 78 |
| Conjugate 3 | 1 | | 100 | 127 | 100 | 44 | 88 | 60 |
| | | N | 5 | 5 | 5 | 5 | 5 | 5 |
| | | Mean | 100 | 113 | 97 | 72 | 96 | 72 |
| Conjugate 3 | 3 | | 100 | 74 | 75 | 51 | 60 | 102 |
| Conjugate 3 | 3 | | 100 | 84 | 140 | 53 | 103 | 89 |
| Conjugate 3 | 3 | | 100 | 80 | 110 | 56 | 85 | 66 |
| Conjugate 3 | 3 | | 100 | 102 | 121 | 58 | 125 | 116 |
| Conjugate 3 | 3 | | 100 | 70 | 127 | 66 | 90 | 93 |
| | | N | 5 | 5 | 5 | 5 | 5 | 5 |
| | | Mean | 100 | 82 | 115 | 57 | 93 | 93 |
| Conjugate 4 | 0.25 | | 100 | 147 | 132 | 142 | 129 | 75 |
| Conjugate 4 | 0.25 | | 100 | 156 | 104 | 93 | 87 | 88 |
| Conjugate 4 | 0.25 | | 100 | 129 | 203 | 108 | 121 | 118 |
| Conjugate 4 | 0.25 | | 100 | 151 | 151 | 133 | 138 | 147 |
| Conjugate 4 | 0.25 | | 100 | 144 | 102 | 90 | 61 | 80 |
| | | N | 5 | 5 | 5 | 5 | 5 | 5 |
| | | Mean | 100 | 145 | 138 | 113 | 107 | 102 |
| Conjugate 4 | 1 | | 100 | 82 | 88 | 79 | 72 | 53 |
| Conjugate 4 | 1 | | 100 | 84 | 131 | 69 | 54 | 68 |
| Conjugate 4 | 1 | | 100 | 90 | 113 | 85 | 94 | 83 |
| Conjugate 4 | 1 | | 100 | 86 | 136 | 86 | 106 | 71 |
| Conjugate 4 | 1 | | 100 | 100 | 159 | 149 | 92 | 123 |
| | | N | 5 | 5 | 5 | 5 | 5 | 5 |
| | | Mean | 100 | 88 | 125 | 94 | 84 | 80 |
| Conjugate 4 | 3 | | 100 | 136 | 79 | 75 | 68 | 48 |
| Conjugate 4 | 3 | | 100 | 124 | 156 | 123 | 157 | 116 |
| Conjugate 4 | 3 | | 100 | 89 | 98 | 92 | 118 | 71 |
| Conjugate 4 | 3 | | 100 | 118 | 66 | 81 | 61 | 53 |
| Conjugate 4 | 3 | | 100 | 124 | 78 | 66 | 119 | 63 |
| | | N | 5 | 5 | 5 | 5 | 5 | 5 |
| | | Mean | 100 | 118 | 95 | 87 | 105 | 70 |

ANGPTL3, angiopoietin-like protein 3; mRNA, messenger ribonucleic acid; N, number.

TABLE 4
Human ANGPTL3 mRNA Expression in Liver On Day
49 In Humanized Mice Treated With A Single Dose
Of Conjugate 2, Conjugate 3 or Conjugate 4

| | | | Human |
|---|---|---|---|
| | | | ANGPTL3 |
| | Dose | | mRNA (2-^{ΔCt} |
| Treatment | (mg/kg) | | vs mRPLO) |
| PBS | 0 | | 7.67 |
| PBS | 0 | | 4.07 |
| PBS | 0 | | 5.06 |
| PBS | 0 | | 6.74 |
| PBS | 0 | | 4.77 |
| | | N | 5 |
| | | Mean | 5.66 |
| Conjugate 2 | 0.25 | | 5.26 |
| Conjugate 2 | 0.25 | | 9.16 |
| Conjugate 2 | 0.25 | | 11.1 |
| Conjugate 2 | 0.25 | | 4.23 |
| Conjugate 2 | 0.25 | | 6.51 |
| | | N | 5 |
| | | Mean | 7.24 |
| | | Knockdown | −27.9 |
| | | (%) | |
| Conjugate 2 | 1 | | 4.48 |
| Conjugate 2 | 1 | | 4.87 |
| Conjugate 2 | 1 | | 5.22 |
| Conjugate 2 | 1 | | 4.73 |
| Conjugate 2 | 1 | | 9.37 |
| | | N | 5 |
| | | Mean | 5.73 |
| | | Knockdown | −1.2 |
| | | (%) | |
| Conjugate 2 | 3 | | 2.81 |
| Conjugate 2 | 3 | | 2.45 |
| Conjugate 2 | 3 | | 2.10 |
| Conjugate 2 | 3 | | 1.93 |
| Conjugate 2 | 3 | | 3.12 |
| | | N | 5 |
| | | Mean | 2.48 |
| | | Knockdown | 56.2 |
| | | (%) | |
| Conjugate 3 | 0.25 | | 7.38 |
| Conjugate 3 | 0.25 | | 5.48 |
| Conjugate 3 | 0.25 | | 4.48 |
| Conjugate 3 | 0.25 | | 3.73 |
| Conjugate 3 | 0.25 | | 5.74 |
| | | N | 5 |
| | | Mean | 5.36 |
| | | Knockdown | 5.3 |
| | | (%) | |
| Conjugate 3 | 1 | | 3.50 |
| Conjugate 3 | 1 | | 6.93 |
| Conjugate 3 | 1 | | 5.59 |
| Conjugate 3 | 1 | | 5.14 |
| Conjugate 3 | 1 | | 5.50 |
| | | N | 5 |
| | | Mean | 5.33 |
| | | Knockdown | 5.9 |
| | | (%) | |
| Conjugate 3 | 3 | | 9.02 |
| Conjugate 3 | 3 | | 8.24 |
| Conjugate 3 | 3 | | 5.13 |
| Conjugate 3 | 3 | | 6.48 |
| Conjugate 3 | 3 | | 7.76 |
| | | N | 5 |
| | | Mean | 7.32 |
| | | Knockdown | −29.3 |
| | | (%) | |
| Conjugate 4 | 0.25 | | 4.57 |
| Conjugate 4 | 0.25 | | 6.16 |
| Conjugate 4 | 0.25 | | 7.89 |
| Conjugate 4 | 0.25 | | 10.65 |
| Conjugate 4 | 0.25 | | 6.84 |
| | | N | 5 |
| | | Mean | 7.22 |
| | | Knockdown | −27.5 |
| | | (%) | |
| Conjugate 4 | 1 | | 8.64 |
| Conjugate 4 | 1 | | 5.74 |
| Conjugate 4 | 1 | | 3.23 |
| Conjugate 4 | 1 | | 5.57 |
| Conjugate 4 | 1 | | 4.68 |
| | | N | 5 |
| | | Mean | 5.57 |
| | | Knockdown | 1.6 |
| | | (%) | |
| Conjugate 4 | 3 | | 4.99 |
| Conjugate 4 | 3 | | 10.68 |
| Conjugate 4 | 3 | | 6.18 |
| Conjugate 4 | 3 | | 1.40 |
| Conjugate 4 | 3 | | 4.24 |
| | | N | 5 |
| | | Mean | 5.50 |
| | | Knockdown | 2.9 |
| | | (%) | |

ANGPTL3, angiopoietin-like protein 3; mRNA, messenger ribonucleic acid; N, number.

### Example 5

### Therapeutic Effects of Conjugate 1 in an Obese HFpEF ZSF1 Rat Model In Vivo.

| Abbreviation or | |
|---|---|
| special term | Explanation |
| ELISA | Enzyme-linked immunosorbent assay |
| GalNAc | Galactose/N-acetylgalactosamine |
| HF | Heart failure |
| HFpEF | Heart failure with preserved ejection fraction |
| HS | High salt |
| IVRT | Isovolumic relaxation time |
| LPL | Lipoprotein lipase |
| LV | Left ventricle |
| NT-proBNP | N-terminal pro B-type natriuretic peptide |
| PBS | Phosphate buffered saline |
| SC | Subcutaneous |
| siRNA | Small interfering ribonucleic acid |
| T2DM | Type 2 diabetes |
| TG | Triglyceride |
| UACR | Urine albumin-creatinine ratio |
| Unx | Uninephrectomized - One kidney removed surgically |
| ZSF-1 | Male obese Zucker diabetic fatty/spontaneously |
| | hypertensive heart failure F1 hybrid (ZSF1) rats |
| | with leptin mutations, when uninephrectomized and |
| | under high-salt diet they develop HFpEF |

A ZSF-1 obese rat model with heart failure with preserved ejection fraction (HFpEF) was used to study the effect of ANGPTL3 protein reduction using Conjugate 1.

Once secreted into the circulation, ANGPTL3 acts as an inhibitor of lipoprotein lipase (LPL), thus, play a major role in regulating total levels of circulating plasma triglycerides (Prog Lipid Res. 2022; 85101140). It is known that similar to the positive metabolic profile in individuals deficient in ANGPTL3 protein (The Journal of clinical endocrinology and metabolism. 2012; 97(7):E1266-75), pharmacological reduction in circulating ANGPTL3 protein has been demonstrated to cause clinically meaningful and well-tolerated reductions in cardiovascular risk factors (Evinacumab. (EVKEEZA®) Prescribing Information. Regeneron Pharmaceuticals, Inc. 2023, Circulation 2019. p. E987-8). Data from observational studies (Arterioscler Thromb Vasc Biol. 2018; 38(2):464-72) has also indicated the potential clinical benefit of such reductions in patients with metabolically driven heart failure.

The ZSF-1 rat is a cross of two different leptin receptor mutations and the offspring can be either obese or lean. The obese ZSF-1 rats spontaneously develop obesity, hypertension, type 2 diabetes, hyperlipidemia, left ventricular (LV) dysfunction, nephropathy and insulin resistance. The lean ZSF-1 rat is hypertensive, but shows none of the other conditions. Obese uninephrectomized ZSF-1 rats were treated for 12 weeks with Conjugate 1 or vehicle respectively, and given high salt diet (to induce HFpEF phenotype). Non-invasive imaging, echocardiography, was used in this study as the “golden standard” of evaluating heart function. It was concluded that ANGPTL3 protein reduction decreases circulating triglyceride (TG) levels, reduces TG content in heart and reduces the development of diastolic dysfunction in ZSF-1 obese rats with HFpEF (as shown by NT-proBNP and IVRT results), without significant increase of liver TGs.

Obese uninephrectomized (Unx) ZSF-1 rats were treated for 12 weeks with Conjugate 1 or vehicle, n=8 respectively, in high salt (HS) diet. Lean ZSF-1 rats (n=8) were used as a control group. Blood samples were taken before start of the treatment and after 12 weeks treatment for analysis of NT-proBNP, ANGPTL3 and circulating lipids and lipoproteins. At termination, tissue from heart, kidney and liver was collected for analysis. Non-invasive imaging, echocardiography, was used in this study as the “golden standard” for evaluating heart function. Isovolumic relaxation time (IVRT) was the primary endpoint.

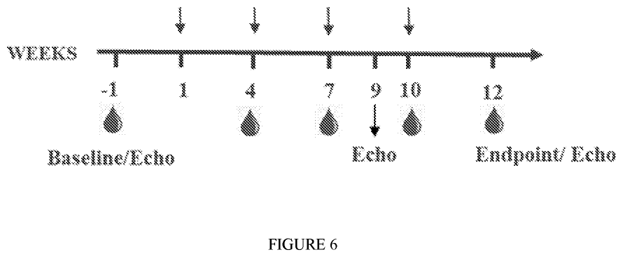

The study design for each rat is outlined in FIG. 6. The rats were grouped 4 and 4 in each cage and were fed pellets containing high salt diet or Purina and normal tap water ad libitum throughout the study. Once every week, the rats were weighed. The rats were randomised into groups by IVRT, glucose, UACR and body weight. Following acclimatization, baseline echocardiography and baseline blood sampling was initiated.

Study design
Dose(s): 4 mg/kg
Infusion/injection rate(s)
   and/or time: Sub cutaneous injections
Duration of treatment: 12 weeks
Number/group: 8, Groups are outlined in Table 5
Number of groups: 7
Individual animal Identification or reference number is the
identification No/reference No: individual identifying number which was given to each animal in the study

TABLE 5
Groups, Doses and Number of Animals

| Group | | | | | Number of | Disease- |
|---|---|---|---|---|---|---|
| number | Group | Treatment | Diet | Dose | animals | population |
| 1 | ZSF1 Lean | PBS | Purina | — | 8 | |
| 2 | ZSF1 Obese | PBS | Purina | — | 8 | |
| | sham | | | | | |
| 3 | ZSF1 Obese | Conjugate 1 | Purina | 4 mg/kg | 8 | |
| | sham | | | | | |
| 4 | ZSF1 Obese | PBS | Purina | — | 8 | CKD |
| | Unx | | | | | |
| 5 | ZSF1 Obese | Conjugate 1 | Purina | 4 mg/kg | 8 | CKD |
| | Unx | | | | | |
| 6 | ZSF1 Obese | PBS | Purina + High | — | 8 | HFpEF |
| | Unx + HS | | Salt | | | |
| 7 | ZSF1 Obese | Conjugate 1 | Purina + High | 4 mg/kg | 8 | HFpEF |
| | Unx + HS | | Salt | | | |

| | |
|---|---|
| Total number of animals | 56 |

The study design for each rat is outlined in FIG. 6. The rats were grouped 4 and 4 in each cage and were fed pellets containing high salt diet or Purina and normal tap water ad libitum throughout the study. Once every week, the rats were weighed. The rats were randomised into groups by IVRT, glucose, UACR and body weight. Following acclimatization, baseline echocardiography and baseline blood sampling was initiated.

Obese uninephrectomized ZSF1 rats on hight salt diet were treated for 12 weeks with Conjugate 1 or vehicle, n=8 respectively. Lean ZSF1 rats (n=8) were used as a control group. Treatments (either PBS or Conjugate 1) were administered subcutaneously at study week 1, 4, 7 and 10. Blood samples were taken before start of the treatment, every third week and after 12 weeks treatment for analysis of NT-proBNP, ANGPTL3 and circulating lipids. At termination, blood was collected as well as tissue from heart, kidney and liver for analysis. Non-invasive imaging, echocardiography, was used in this study as the “golden standard” of evaluating heart function. Isovolumic relaxation time (IVRT from echocardiography), was the primary endpoint. Animals N 25 (Obese Unx+HS+AD) 30 and 32 (Obese Unx+HS) died before endpoint.

### Experimental Procedures

Blood Sampling from Tail Vein

At baseline, every third week and endpoint (as described in FIG. 1), a tail vein blood sample was collected from the wake rats via a butterfly catheter (21G, BD Valu-Set™, Plymouth, UK) in EDTA coated vials (MiniCollect, K3EDTA, Kremsmünster, Austria).

### Plasma Analysis

Blood was centrifuged for 10 min at 5000×g. EDTA-plasma and was used for analysis of NT-proBNP and ANGPTL3. The assays were performed according to the manufacturer's instructions.

### Metabolic Cages

Animals were place in metabolic cages urine sampling and fasting period for later blood collection. From an animal welfare perspective, a shorter time, such as 4 hours, is recommended as it is less challenging for the rats (less stressful and less impact on body weight, food, and water intake). At study weeks-1, 7, 10 and 12, animals were placed in metabolic cages for 4 hours with water ad libitum but no food. For the fasting period of the blood collection of week 4, animals were placed in a plastic box for 4 hours with water ad libitum.

### Echocardiography

### Animal Preparation Echocardiography

Non-invasive imaging, Echocardiography, was performed three times: at baseline, week 9 and study week 12. The animals were initially anaesthetised with isoflurane (Isoba® Vet, Schering-Plough Ltd., England) gas (5%) in a gas chamber. The gas concentration was established by exerting a flow of 02 (200 mL/min) and air (500 mL/min) to a solution of isoflurane. During the experiment the animals were kept anaesthetised by breathing isoflurane gas (approximately 2.2%) through a mask. Hair removal of the chest and left back side is done with Veet® removal cream after shaving. The skin was carefully rinsed with lukewarm tap water.

### Experimental Protocol Echocardiography

Echocardiography was performed using a high-resolution ultrasound scanner (Vevo 3100, Visualsonics Inc) with MX201 solid state transducers. A parasternal long axis (LAX) view was used to visualize a long axis image of the heart. Subsequently a short-axis (SAX) image was collected at the mid-ventricular level, with the right ventricle (RV) oriented to the left side of the field of view (FOV), and the papillary muscles on the right side. B-mode and m-mode loops were collected.

To assess diastolic function including isovolumic relaxation time (IVRT), analyses of the blood flow spectrum from a pulse wave Doppler on the mitral valve were performed. This image was acquired via a modified apical four chamber view. From the same view Tissue Doppler Imaging (TDI) was performed on the septal mitral valve annulus to assess longitudinal velocities of the myocardium.

### Termination and Organ Sampling

At endpoint, the heart, lungs, spleen, liver and one kidney were harvested and weighed and the tibia was dissected and the length was measured.

### Rat ANGPTL3 ELISA

This method was set up fully automated in a Beckman Coulter Motoman robotic system (FLIPR). Including Biomek FX Span-8/96-head Biotek cellwasher, Cytomat Incubators (shake), Carousel and MD Paradigm Reader. Assay kit Mouse/Rat Angiopoietin-like protein 3 ELISA (ANGPTL3, Cat No RAG011R, Czech Republic) was used.

Robot assay: Samples were pre-diluted in two steps from 10 μL Sample, to in total 800× (separate method). Standards were serial diluted (2×) and transferred to predilution plate. 100 μL prediluted sample/standard were pipetted to ELISA plate. Incubation for 1 h at 37° C. ELISA plate was washed (ELISA3 5× washes). 100 μL detection antibody was added. Incubation for 1 h at 37° C. ELISA plate was washed (ELISA3 5× washes). 100 μL HRP Conjugate was added. Incubation for 1 h at 37° C. ELISA plate was washed (ELISA3 5× washes). 100 μL Substrate TMB was added. Incubation for 15 min at room temperature in the dark. 100 μl top solution was added. Absorbance at 450 nm and reference wavelength at 540 nm (within 20 min) was read.

### Rat NT-proBNP MSD ELISA Assay

This method was set up fully automated in a Beckman Coulter SCARA robotic system (Lambda) including Biomek NX Span-8, Biomek FX 96/96-heads, biotek cellwasher, cytomat Incubators, hotels and MD Paradigm Reader and MSD S600. Rat NT-proBNP kit (Cat No K153JKD-1) was used.

Robot assay: Samples were pre-diluted (3×) in Diluent 34 with protease inhibitor. MSD plate was washed (3× in wash buffer). 50 μL of standards, controls and diluted sample were dispensed. Incubation for 1 h with vigorous shaking (500 rpm) at room temperature. Plate was washed (3× in wash buffer). 25 μL detection antibody was dispensed. Incubation for 2 h with vigorous shaking (500 rpm) at room temperature. Plate was washed (3× in wash buffer). 150 μL reading buffer was dispensed. Plate was read within 2 min in the MSD Sector Imager S600.

### Straight Phase HPLC for Plasma TGs

Plasma triglycerides were extracted using liquid/liquid extraction (Lofgren L, 2012). The liquid extract was evaporated under a stream of nitrogen and reconstituted in heptane: isopropanol [9:1]. The analysis was done using straight phase HPLC as described previously (J Chromatogr B Biomed Sci Appl. 1998; 708(1-2): 21-6). Quantification was made against an external calibration curve.

### TG Content in Liver

Pieces (app 0.50 mg) of liver were homogenized with isopropanol (2-isopropanol) so the triglycerides is released from the tissue. Triglycerides in sample were hydrolyzed enzymatically to glycerol and fatty acids. Glycerol generates, by two enzymatic steps, hydrogen peroxide that contributes to the formation of a red quinoneimine derivat. Colour intensity is directly proportional to the amount of triglycerides (glycerol) in the sample, and is measured colorimetrically at wave length 500 nm.

### Lipidomics for TG Content in the Heart

Pre-weighed frozen ZSF1 heart samples were homogenized by bead beating in 10× (v/w) 3:1:6 IPA:water:EtOAc containing 1:100 EquiSplash (Avanti) internal standard mixture. A fraction of the clarified supernatant was dried via speedvac before resuspension in an equal volume of 9:1 MeOH: Toluene (10× ratio maintained). 1 μL of sample was analyzed using a C18 LCMS-based approach.

Mass Spectrometry: Untargeted (full scan) mass spectrometry analysis was performed using Thermo Tribrid ID-X with a scan range of 50 to 950 m/z for metabolomics and 50 to 1700 m/z for lipidomics. Individual samples were acquired in MS1 only mode, using a polarity switching approach.

Data analysis: Thermo raw files were analyzed in MS-DIAL (≥v. 4.8) for feature detection, alignment, identification, and normalization. Peak areas were normalized to EquiSplash standards for lipidomics and to the total ion current for metabolomics. Features were filtered in R based on QC/Blank signal >3× and a QC RSD <30%. Statistical analysis was performed using the MetaboAnalystR package (v.3.2.0) with R language (v.4.1.0). Briefly, data was normalized by median, log-transformed and scaled with a Pareto approach. Fold change calculations are using data before the column-wise normalization was applied (ie at the original scale).

### Proteomics

Proteomics data aquisition: Mouse heart tissues were lysed in metabolite extraction buffer (IPA:H2O:EtOAt=6:1:3) and beads beat, protein pellets with leftover buffer will be used for proteome profiling. High pH Reversed-Phase Peptide Fractionation (Thermo 84868) was performed for the peptide fractionation. LC-MS/MS analysis was conducted on a timsTOF Pro mass spectrometer (Bruker) coupled with a Evosep LC-system and nano-electrospray ion source (CaptiveSpray Source, Bruker). Data-dependent acquisition (DDA) and Data-independent acquisition (DIA)-PASEF mode were performed to obtain the peptide data. The combined DDA and DIA acquisition raw files were analyzed via Spectronaut (Biognosys AG) software (Version 18) with Pulsar search engine (SN14.10.201222) to build the library, using UniProt rat proteome database (UP000002494_20231024).

Proteomics data analysis: Protein intensities were analyzed using R (v. 4.2.3) and DEP package (v.1.20.0). Briefly, the data was normalized using svm approach, and missing values were imputed using missForest approach (v.1.5). Finally, differential analysis was done using linear models and empirical Bayes statistics. PCA was calculated using the imputed values. Enrichment analysis was performed using clusterProfiler package (v.4.6.2).

### Electron Microscopy for Podocyte Health

A thin slice of the kidney was fixed in a mix of 2.5% glutaraldehyde+1% paraformaldehyde (both from Sigma-Aldrich) in 0.1 M phosphate buffer (pH 7.4) and stored at 4° C. Electron microscopy analysis was conducted at the electron microscopy unit at Huddinge University Hospital (Stockholm, Sweden).

### Data Analysis

Holm-Sidak's multiple comparisons test or Unpaired t-test was used for statistical evaluation. Data is presented as mean±SD. In other cases it is indicated in the footnotes of the figures.

Histology readouts: Glomerular score, Podocyte stress and TEM (Glomerular Basement Membrane, GBM, thickness and slit/μm).

Assays used for analyzing urine samples: U-Albumin and U-Creatinine are measured in Pentra 400. U-Albumin is determined colorimetric: Albumin CP ABX Pentra, ref A11A01664. U-Creatinine is determined by Jaffe's reaction (picric acid): Creatinine 120CP, Horiba ABX A11A01933. U-Albumin and creatine volume required for analyses around 50 μL.

### Results

### Body Weight

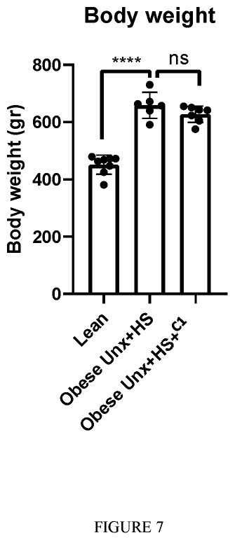

As seen in FIG. 7, changes in body weight between lean and obese rats were observed. No change in body weight was observed in obese animal treated with Conjugate 1 (C1) compared to obese control group. Filled-black circles represent individual animals. Stars indicate significant changes between the comparison groups. Not significant (ns) indicates no significant changes between the comparison groups. Holm-Sidak's multiple comparisons test was used. Lean (N=8), Obese Unx+HS (N=6) Obese, Unx+HS+C1 (N=7). Lean-control animals, Obese Unx+HS-uninephrectomy+high-salt diet to induce HFpEF, Obese Unx+HS+AD, uninephrectomy+high-salt diet to induce HFpEF+treatment with Conjugate 1.

### ANGPTL3 Protein and TG Plasma Levels

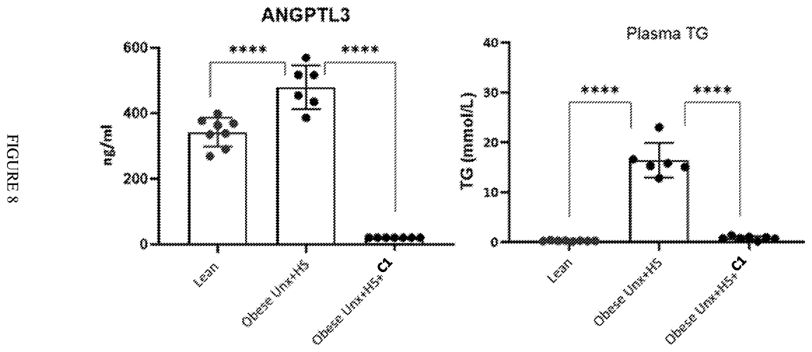

As seen in FIG. 8, Conjugate 1 reduced more than 95% of ANGPTL3 circulating protein levels in the Obese Unx+HS+AD group compared to the control group. This changes were also observed in the reduction of circulating plasma triglyceride (TG) levels that were reduced almost to baseline levels after treatment. Filled-black circles represent individual animals. Stars indicate significant changes between the comparison groups. Holm-Sidak's multiple comparisons test was used. Lean (N=8), Obese Unx+HS (N=6) Obese, Unx+HS+AD (N=7).

Lean-control animals, Obese Unx+HS-uninephrectomy+high-salt diet to induce HFpEF, Obese Unx+HS+AD, uninephrectomy+high-salt diet to induce HFpEF+treatment with Conjugate 1.

### TG Content (Lipidometic Analysis) in ZSF-1 Rat Hearts and Liver

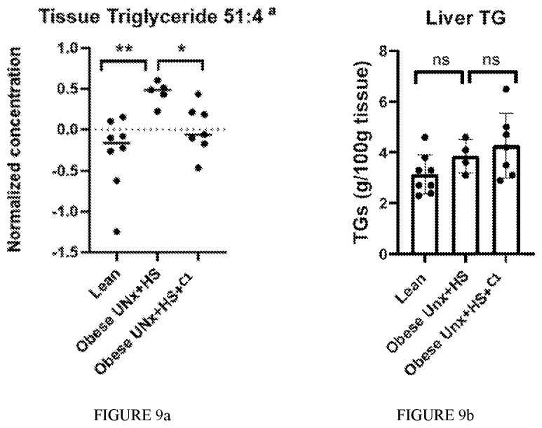

FIG. 9a shows a significant reduction in TG content (lipidomic analysis) in heart tissue by Conjugate 1 in the Obese Unx+HS+AD group compared to control group Tissue triglyceride 51:4, 51 carbons, 4 double bonds). As seen in FIG. 9b, ANGPTL3 gene silencing does not affect liver TG content in the Obese Unx+HS+AD group compared to comparison group. Filled-black circles represent individual animals. Stars indicate significant changes between the comparison groups. Not significant (ns) indicates no significant changes between the comparison groups. Holm-Sidak's multiple comparisons test was used. Lean (N=8), Obese Unx+HS (N=6) Obese, Unx+HS+C1 (N=7). Lean-control animals, Obese Unx+HS-uninephrectomy+high-salt diet to induce HFpEF, Obese Unx+HS+AD, uninephrectomy+high-salt diet to induce HFpEF+treatment with Conjugate 1.

### NT-proBNP and IVRT

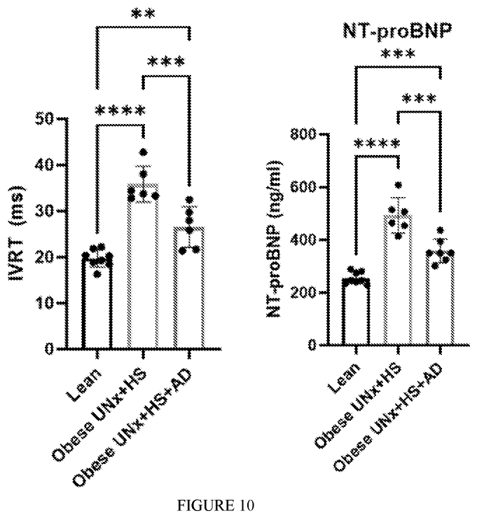

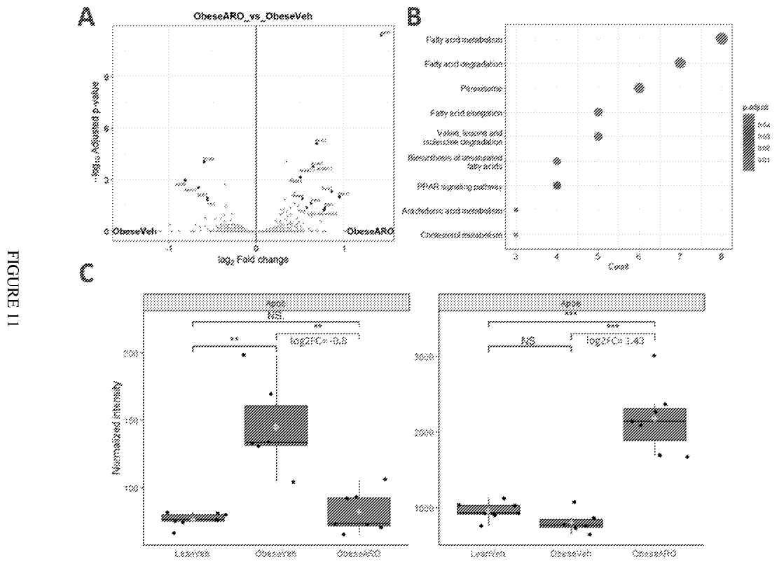

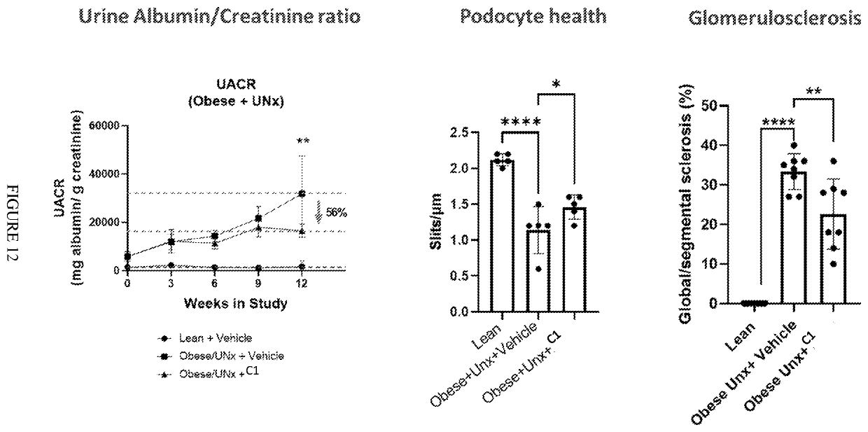

At a functional level, prevention of cardiac diastolic dysfunction was demonstrated by decreased isovolumic relaxation time and circulating N-terminal pro B-type natriuretic peptide levels (FIG. 10). NT-proBNP is a biomarker for strain in the heart muscle and isovolumic relaxation time is an echocardiography diastolic function parameter. Filled-black circles represent individual animals. Stars indicate significant changes between the comparison groups. Not significant (ns) indicates no significant changes between the comparison groups. Holm-Sidak's multiple comparisons test was used. Lean (N=8), Obese Unx+HS (N=6) Obese, Unx+HS+C1 (N=7). Lean-control animals, Obese Unx+HS-uninephrectomy+high-salt diet to induce HFpEF, Obese Unx+HS+AD, uninephrectomy+high-salt diet to induce HFpEF+treatment with Conjugate 1.

### Proteomics Data (Heart Tissue)

Proteomics data from heart tissue showed changes in proteins involved in Fatty acid metabolism. This was confirmed in using several pathway platforms. Specifically, ANGPTL3 inhibition altered the concentration of several Apolipoproteins. This results suggest that the treatment enhances the lipid degradation in heart tissue.

A) Volcano plot showing proteins changing significantly (Adj p-value <0.1 &|log 2 (FC)|>0.5). B) KEGG pathways significantly enriched. C) Levels of Apolipoproteins in the different treatment groups (“***”=0.001, “**”=0.01, “*”=0.05). Lean-control animals, Obese Unx+HS-uninephrectomy+high-salt diet to induce HFpEF, Obese Unx+HS+ARO, uninephrectomy+high-salt diet to induce HFpEF+treatment with Conjugate 1.

### Renal Health

As shown in FIG. 8, ANGPTL3 inhibition prevents kidney damage in ZSF-1 rats with CKD, represented by UACR, podocyte injury, and glomerulosclerosis. The rats used for the CKD indication were not under high salt diet. They received purina diet for the duration of the study. Filled-black circles, triangles and squares represent individual animals. Stars indicate significant changes between the comparison groups. Holm-Sidak's multiple comparisons test was used. N number varies depending on the assay performed.

Lean-control animals, Obese Unx-uninephrectomy to induce CKD, Obese Unx+ARO, uninephrectomy to induce CKD+treatment with Conjugate 1.

Treatment with Conjugate 1 showed that reduction in ANGPTL3 protein improves cardiometabolic outcomes in ZSF-1 HFpEF rats by decreasing circulating TGs levels, reducing TG content in heart and reducing the development of diastolic dysfunction.

It is hypothesized that targeting ANGPTL3 mRNA and consequently silencing hepatic ANGPTL3 gene expression with Conjugate 2, or any other siRNA agent for inhibition of ANGPTL3, will enhance LPL enzymatic activity, promote hydrolysis of TG-rich lipoproteins, decrease circulating TG and improve cardiac and renal outcomes.

### Example 6

Therapeutic Effects of Conjugate 1 in Cynomolgus Macaques (Macaca fascicularis) with HFpEF.

The intention of this study was to investigate the chronic effects of the target inhibition of the Conjugate 1 on cardiac-renal functional measures and markers in the most relevant animal model that reflects the intended human population. The cynomolgus monkey was selected as the test species of choice over other lower mammalian species for more meaningful and translatable results because it is closely related to humans, both phylogenetically and physiologically. The chronic efficacy of Conjugate 1 in the treatment of heart failure was explored in the cynomolgus monkey model with heart failure with preserved ejection fraction (HFpEF) which closely approximates the adult human HFpEF phenotype.

### Study Design

Group 1: Age-matched normal healthy control (4 subjects)
Group 2: HFpEF PBS control (10 subjects)
Group 3: HFpEF Conjugate 1 s.c. dose 3 mg/kg (10 subjects)
Group 4: HFpEF Empagliflozin p.o. dose 3 mg/kg (10 subjects)
Dosing was administered on day 1 of week 1 and day 84 of week 12. This was then followed by a 12 week post-dose observation/washout period.

### Diet and Water

The monkeys were provided 3 meals per day consisting of 100.0 g of standard monkey formula feed (extruded pellets) in the morning 9:00-10:00 AM, one regular apple (150 g) in the afternoon 14:00-15:00 PM, and 100 g of high-fat diet (HFD) in the evening 16:00-17:00 PM. Water was provided ad libitum. All the monkeys were fasted overnight. After each feeding time, all remaining food was withdrawn, and food intake determined by weighing the left-over food.

### Two-Dimensional (2D) Echocardiography

Two-dimensional echocardiography (2D echo) was performed using the Mindray M9CV

Echocardiography System (Mindray, Shenzhen China) with SP5-1s (1.1-4.4 MHz) phased array transducer under ketamine sedation administered intramuscularly (IM) at 10 mg/kg.

Animals were fasted for 4-6 hours to minimize the risk of vomiting and lung aspiration during anaesthesia induction and recovery. Nevertheless, unlimited access to drinking water was provided during the fasting period. Ketamine was administered intramuscularly at 10 mg/kg and the hairs on the animal's left chest was shaved thoroughly using an electric clipper and a shaving blade. The monkey were appropriately positioned on the exam table or surgical bed that has been cleaned by wiping with 70% alcohol and covered with sterile disposable drape. The animal was be kept warm on a heating pad/blanket during the entire procedure. Typically, the echocardiogram was performed with the animal in the left lateral decubitus.

### MRI Scan Preparations

Prior to each scan, the monkey was fasted overnight or for a minimum of 6 hours prior to imaging.

Ketamine (5-10 mg/kg, IM) was injected intramuscularly to induce sedation and atropine hydrochloride (0.02-0.04 mg/kg, IM) was administered to inhibit respiratory spasm and salivation. Endotracheal intubation was then carried out, an intravenous (IV) line was inserted into a peripheral vein, and the ECG leads were attached to the animal's chest wall. The animal was then transferred to the MRI bed for ECG and vital signs monitoring. It was connected to the mechanical ventilator and Isoflurane (1-3%) in medical air was used to induce and maintain anesthesia. The animal was positioned supine, head first on the MRI bed and an abdominal coil was used to image the heart. Each scan session did not exceed four (4) hours. The animal was kept warm during the scan. The heart rate, blood oxygenation level, noninvasive blood pressure, respiratory rate, end-tidal C02 level and temperature was recorded/monitored during the entire scan session to ensure adequate level of anesthesia. ECG monitoring was continuously carried out.

### MRI Procedures

All animals underwent CMR using a 3.0 Tesla magnetic resonance imaging system (Magnetom Prisma; Siemens Healthineers, Erlangen, Germany) with a 32-channel phasearray body coil. The following MRI Scan Sequences was carried out twice during the entire study (at baseline and on Week 25): Proton density fat fraction (PDFF) Imaging; Regadenoson Stress Perfusion Imaging; and Extracellular volume (ECV) Scan. The pulse sequence parameters were documented and described in the MRI report.

The animals were mechanically ventilated at 20-30 breaths per min during the scan.

Inspiratory breath holding was carried out to mitigate the effect of respiration on the vascular image quality. This was done only by switching the ventilator off for no more than 10 seconds while running the pulse sequence. The oxygen flow during breath holds (when the ventilator is off) was maintained.

### Proton-Density Fat Fraction (PDFF) Imaging

Myocardial proton density fat fraction (mPDFF) was measured by using a 2D, ECGtriggered, multi-echo segmented gradient-echo sequence. The ECG trigger delay was to ensure data acquisition occurring at the quiescent period of cardiac cycle to minimize cardiac motion. The respiratory motion was controlled by a single breath-hold. The mPDFF was measured using the software, VivoQuant (V2.5), by placing region-of-interest (ROI) at different myocardial locations.

### Regadenoson Stress Perfusion Imaging and Extracellular Volume (ECV) Scan

To perform stress imaging, a rest first-pass perfusion imaging was first be initialized with a bolus injection of MRI contrast agent (0.075 mmol/kg of Gadoteric Acid Meglumine Salt Injection; 15 ml: 5.654g (a sterile solution containing 377 mg/ml gadoteric acid meglumine salt; Jiangsu Hengrui Pharmaceutical Co., Ltd.). The perfusion sequence was a 2D saturation recovered single-shot gradient-echo sequence with 70 repetitions. A total of 3 slices as those in pre-contrast T1 mapping were scanned with a temporal resolution of one RR-interval (˜500 msec). Cine images along 2-, 3-, and 4-chambers were then acquired after the perfusion images. Approximately 15 min later, Regadenoson (5 μg/kg; Nanjing HaiRong Pharmaceutical LTD) was injected in a bolus to induce vasodilation and another first-pass perfusion imaging (0.075 mmol/kg of Gadoteric Acid Meglumine Salt Injection) was performed at 1 min after the injection of Regadenoson for stress perfusion imaging. Ten minutes after the second contrast injection, post-contrast MOLLI T1 mapping was acquired with a scheme of 4(1)3(1) in the same three short-axis slices that are used for the pre-contrast T1 scans. Late gadolinium enhancement images were obtained using an electrocardiogram-gated breath-hold inversion recovery Turbo FLASH at 15 min after contrast injection. All CMR images were prospectively analyzed. A blood sample was obtained from all monkeys immediately before each CMR study for hematocrit (Hct) measurements. Cine images were analyzed by Medis software (Medis Medical Imaging Systems BV, Leiden, Netherlands).

Based on the pre-contrast and post-contrast T1 maps, which were automatically generated with a prototype inline process function from the MRI system, global or regional ECV values were calculated using Hct values. If there was motion between the pre- and post-T1 series, the pre- and post-MOLLI was registered manually. The first-pass perfusion imaging was analyzed by using another custom-made software Perfusion Imaging Toolkit (PIT).

Quantitative perfusion maps in 3 slices were generated for both rest and stress conditions. Myocardial perfusion reserves were then calculated as the ratio of stress perfusion over resting perfusion.

### Blood Sampling for Pharmacokinetic (PK) Profiling, Target Biomarkers and Other Exploratory Pharmacodynamic (PD) Biomarkers

Approximately 6 mL of whole blood samples was collected from a peripheral vein of the non-sedated/alert monkeys directly into K2EDTA tubes for the following target biomarkers. EDTA plasma was separated by centrifugation at 2,500×g for 10 minutes at 4° C. The collected plasma was aliquoted into cryotubes.

The samples were stored frozen at −80° C. The cryotubes were marked with study number, animal number group number, experimental day number and time after dose.

One aliquot for PK was analyzed by KBI and one was stored frozen at −80° C. as backup. Samples for target engagement biomarkers, ANGPTL3, TG were stored and analyzed.

### Proton-Density Fat Fraction (PDFF) Imaging

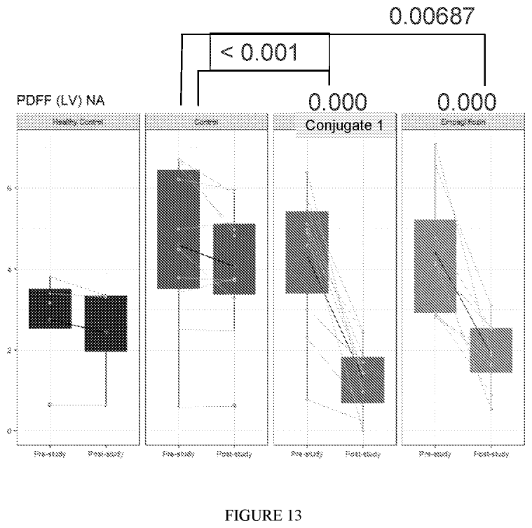

As seen in FIG. 13, animals treated with Conjugate 1 (ANGPTL3 SiRNA inhibition) had signific reduction in heart fat fraction. Similar results were observed with the positive control SGLT2 inhibitor (Empaglifozin) as expected.

### Extracellular Volume (ECV) Scan

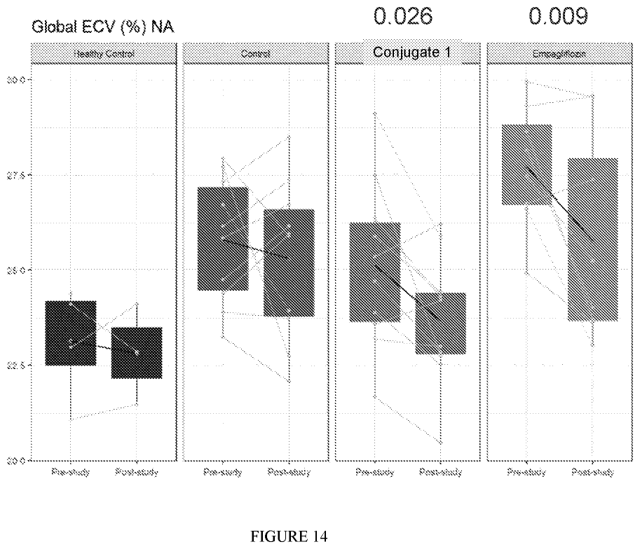

As seen in FIG. 14, ANGPTL3 inhibition significantly reduced extracellular volume. This parameter has been associated with survival rate in heart failure patients.

### Left Atria Volume Index

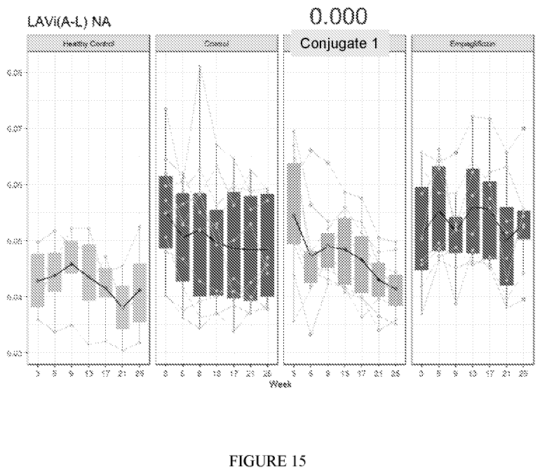

Left atria volume index (LAVi) is a critical parameter for the diagnosis of HfpEF and predicts poor prognosis in these patients (J Cardiovasc Med (Hagerstown). 2018 June; 19 (6): 304-309). As seen in FIG. 15, ANGPTL3 inhibition, but not SGLT2 inhibition, resulted in a significant decrease of LAVi.

### Plasma ANGPTL3 Levels

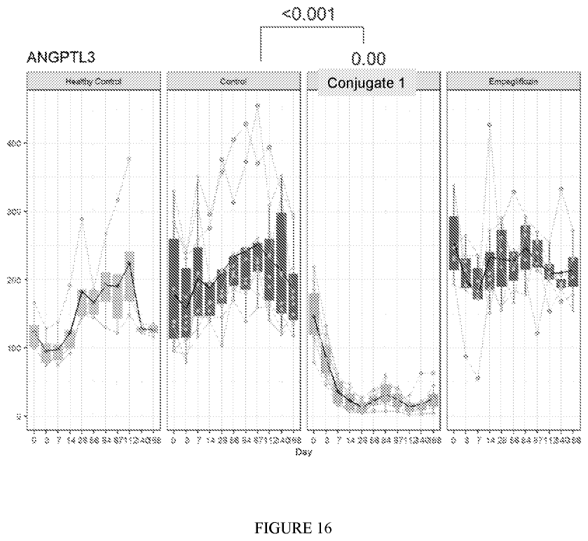

As seen in FIG. 16, ANGPTL3 circulating levels decreased significantly with treatment.

### Plasma Triglyceride (TG) Levels

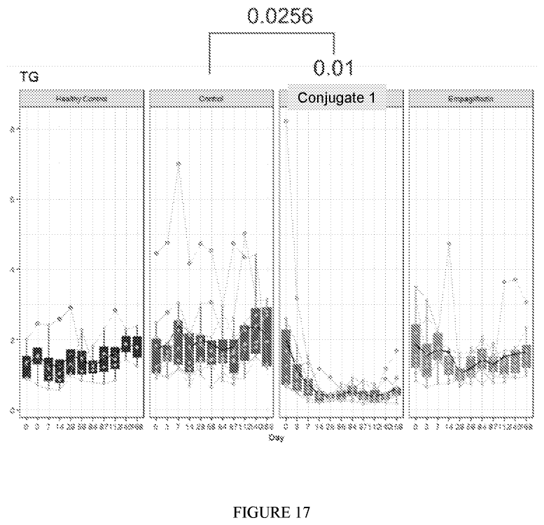

As a consequence of ANGPTL3 inhibition, TG circulating levels were significantly decreased as seen in FIG. 17. Treatment with SGLT2 inhibitor Empaglifozin did not have any effects in reduction of TG circulating levels.

1. An angiopoietin-like 3 (ANGPTL3) siRNA agent comprising (i) a sense strand having the sequence:
[M]-mC mU mA mC mA mU fA fU fA mA mA mC mU mA mC mA mA (ps) mG (ps) mU (SEQ ID NO: 11),
and (ii) a antisense strand having the sequence:
mA (ps) fC (ps) mU fU mG fU mA fG mU fU mU fA mU fA mU fG mU (ps) fA (ps) mG (SEQ ID NO: 5),
where [M] is a triantennary ligand moiety linked to the 5′ end of the sense strand and has the structure: 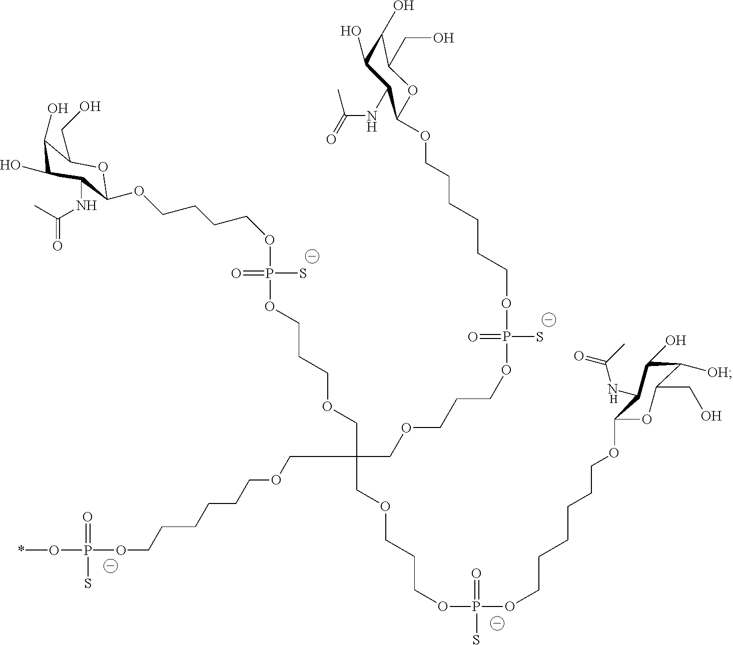
or a pharmaceutically acceptable salt thereof.

2. (canceled)

3. A pharmaceutical composition comprising the ANGPTL3 siRNA agent of claim 1, or a pharmaceutically acceptable salt thereof.

4. A method of treating, or reducing the risk, of a cardiometabolic disease comprising administering to a person suffering from or at risk of said disease, a therapeutically effective amount of the ANGPTL3 siRNA agent of claim 1, or a pharmaceutically acceptable salt thereof.

5. The method of claim 4, wherein said cardiometabolic disease is hypertriglyceridemia, obesity, hyperlipidemia, abnormal lipid and/or cholesterol metabolism, atherosclerosis type I, diabetes mellitus, cardiovascular disease, coronary artery disease, non-alcoholic steatohepatitis, non-alcoholic fatty liver disease, homozygous and heterozygous familial hypercholesterolemia, or statin resistant hypercholesterolemia.

6-18. (canceled)

19. The pharmaceutical composition of claim 3, wherein the composition comprises an admixture with a pharmaceutically acceptable adjuvant, diluent, or carrier.

20. A method of treating, or reducing the risk, of a cardiometabolic disease comprising, administering to a person suffering from or at risk of said disease, a therapeutically effective amount of the pharmaceutical composition of claim 3, or a pharmaceutically acceptable salt thereof.

21. The method of claim 20, wherein said cardiometabolic disease is hypertriglyceridemia, obesity, hyperlipidemia, abnormal lipid and/or cholesterol metabolism, atherosclerosis type I, diabetes mellitus, cardiovascular disease, coronary artery disease, non-alcoholic steatohepatitis, non-alcoholic fatty liver disease, homozygous and heterozygous familial hypercholesterolemia, or statin resistant hypercholesterolemia.
